(19) United States
(12) Patent Application Publication
(10) Pub. No.: US 2024/0279617 A1
(43) Pub. Date: Aug. 22, 2024

(54) BIOMIMETIC SYSTEMS AND DISEASE ANALYSIS METHODS

(71) Applicants: The Johns Hopkins University, Baltimore, MD (US); UNIVERSITY OF WASHINGTON, Seattle, WA (US)

(72) Inventors: Deok-Ho Kim, Seattle, WA (US); Chia-Yi Su, Baltimore, MD (US); Alice Burchett, Deer Park, WA (US); Marcus Williams, Baltimore, MD (US)

(21) Appl. No.: 18/566,519

(22) PCT Filed: Jun. 6, 2022
(86) PCT No.: PCT/US22/32404

§ 371 Date: Dec. 1, 2023

Related U.S. Application Data
(60) Provisional application No. 63/197,319, filed on Jun. 4, 2021.

(51) Int. Cl. C12N 5/09; C12M 3/06; G01N 33/50

(52) U.S. Cl. CPC C12N 5/0693; C12M 27/14; G01N 33/5011; C12N 2503/02; C12N 2533/54

(57) ABSTRACT

Assay and systems are provided that include use of cells and/or organoid for patient diagnosis, drug development, and personalized medicine, including for diagnosis and treatment of cancer and associated diseases and disorders. In one aspect, systems and methods include applying a phase-specific and force-guided polymerization of a biomaterial, wherein the polymerization comprises a nucleation phase and an elongation phase; thereby, producing a model.

The present application claims the benefit of and priority to U.S. provisional application No. 63/197,319 filed Jun. 4, 2021, which is incorporated herein by reference in its entirety.

### BACKGROUND

Prior in vitro tumor models suffer from a number of notable shortcomings.

### BRIEF SUMMARY

In one aspect, provided are assays and methods for analysis and cancer therapies selected for treatment of particular cells.

In one aspect, assays and systems are provided that include use of a tumor cell and/or organoid for patient diagnosis, drug development, and personalized medicine, including for diagnosis and treatment of cancer and associated diseases and disorders.

We now provide systems and methods to produce a cellular or tissue model, comprising: applying a phase-specific and force-guided polymerization of a biomaterial, wherein the polymerization comprises a nucleation phase and an elongation phase; thereby, producing a model.

In one aspect, we now provide a tumor model (including a three-dimensional (3D) biomimetic tumor model) comprising: one or more tumor cells and/or organoids embedded or otherwise associated a matrix comprising radially aligned fibers and circumferentially oriented or aligned fibers.

Preferred cells and organoids will comprise a matrix region that substantially comprises radially aligned fibers and a distinct matrix region that substantially comprises circumferentially oriented or aligned fibers.

Tumor cell or organoid regions that comprise radially aligned fibers (particularly matrix fibers) suitably will comprise radially aligned fibers in varying amounts including in certain systems suitably from 10 to 90 percent of the total volume of matrix material that surrounds or is otherwise associated with the cells or organoid will be substantially radially aligned fibers and the balance will include a region that substantially comprises circumferentially oriented or aligned fibers. In certain systems, tumor cell or organoid regions that comprise radially aligned fibers (particularly matrix fibers) will constitute from 30, 40, 50, 60, or 80 percent, or from 40, 50 or 60 percent, of the total volume of matrix material that surrounds or is otherwise associated with the cells or organoid and the balance will include a region that comprises circumferentially oriented or aligned fibers. In areas where the regions of radially aligned fibers and regions circumferentially oriented fibers mate or interface, there may be a gradation and mixing of the differently aligned fibers. As referred to herein, a region that substantially comprises radially aligned fibers will have at least 55, 60, 70, 80 or 90 weight percent of the total fibers of the region radially aligned. As referred to herein, a region that substantially comprises circumferentially oriented or aligned fibers will have at least 55, 60, 70, 80 or 90 weight percent of the total fibers of the region circumferentially oriented or aligned.

Suitably, the tumor cells or organoids are preferably associated with a matrix that comprises radially aligned fibers on one side of a test system (e.g. the organoid) and circumferentially oriented or aligned fibers the opposing side of the test system (e.g. the organoid).

In a further aspect, a biomimetic model is providing (including a three-dimensional (3D) biomimetic model) comprising cells or tissue organoids derived from kidney, heart, liver, brain, lung or stomach embedded or otherwise associated a matrix comprising radially aligned fibers and circumferentially oriented or aligned fibers.

Preferred cells and organoids comprising cells or tissue organoids derived from kidney, heart, liver, brain, lung or stomach will comprise a matrix region that substantially comprises radially aligned fibers and a distinct matrix region that substantially comprises circumferentially oriented or aligned fibers.

Cells or tissue organoids derived from kidney, heart, liver, brain, lung or stomach are preferably associated with a matrix that comprises radially aligned fibers on one side of a test system (e.g. the organoid) and circumferentially oriented or aligned fibers the opposing side of the test system (e.g. the organoid).

Cells or tissue organoids derived from kidney, heart, liver, brain, lung or stomach comprise radially aligned fibers (particularly matrix fibers) will comprise radially aligned fibers in varying amounts including in certain systems suitably from 10 to 90 percent of the total volume of matrix material that surrounds or is otherwise associated with the cells or organoid and the balance will include a region that comprises circumferentially oriented or aligned fibers. In certain systems, cells or tissue organoids derived from kidney, heart, liver, brain, lung or stomach that comprise radially aligned fibers (particularly matrix fibers) will constitute from 30, 40, 50, 60, or 80 percent, or from 40, 50 or 60 percent, of the total volume of matrix material that surrounds or is otherwise associated with the cells or organoid and the balance will include a region that substantially comprises circumferentially oriented or aligned fibers. In areas where the regions of radially aligned fibers and regions circumferentially oriented or aligned fibers mate or interface, there may be a gradation and mixing of the differently aligned fibers. As discussed, as referred to herein, a region that substantially comprises radially aligned fibers will have at least 55, 60, 70, 80 or 90 weight percent of the total fibers of the region radially aligned. As referred to herein, a region that substantially comprises circumferentially oriented or aligned fibers will have at least 55, 60, 70, 80 or 90 weight percent of the total fibers of the region circumferentially oriented or aligned.

Fiber orientation as referred to herein can be readily determined including by second-harmonic generation and computational segmented images and scanning electron microscopic imaging as demonstrated herein.

Tumor cells or organoid with an encasing fiber matrix having different fiber alignment (particularly a first region of radially aligned fibers and a second regions of circumferentially aligned fibers) can be readily prepared by a phase-specific, force-guided method as disclosed herein which may include in preferred aspects by applying two different forces designed for the two phases of fiber polymerization, nucleation and elongation phases. The two different forces may include 1) laminar flow or force (e.g. horizontal laminar Couette flow) in nucleation phase and 2) gravitational force in elongation phase.

Preferably, the matrix comprises collagen I, collagen IV, Matrigel, poly L-lysine, Geltrex, gelatin, nitrogen, fibronectin, fibrinogen, gelatin methacrylate, fibrin, silk, pegylated gels, collagen methacrylate, decellularized extracellular matrices, basement membrane proteins, or any other biomaterial, or a combination thereof.

In particular aspects, the matrix comprises collagen I.

The system or model also suitably may comprise additional materials such as for example cytokines, growth factors, cytotoxic agents, chemotherapeutic agents, differentiation factors, colony stimulating factors (CSFs), interferons, interleukins, chemotactic factors and combinations thereof.

In another aspect, a coaxial rotating cylinder system is provided and may comprise: an outer cylinder; an inner cylinder, wherein the outer and inner cylinders comprise different radii and are concentrically aligned; and preferably a rod (e.g. brass rod) and bearings (e.g. plastic bearings).

In one configuration, suitably the outer cylinder is held fixed by a base and the inner cylinder is free to rotate about its axis. Suitably, the rotation of the inner cylinder is controlled by a rotator apparatus. Suitably, the inner cylinder is connected to the rotator apparatus. Preferably, the cylinder having a smaller radius is inserted into the center of the cylinder having a larger radius. Suitably, the inner cylinder is inserted into the outer cylinder after concentrically aligning the inner and outer cylinders. Suitably, the concentrically aligned cylinders comprise an empty annulus between the two cylinders. Suitably, a biomaterial comprising one or more cells, spheroids or organoids is introduced into the annulus.

In a further aspects, methods are provided for producing a three-dimensional (3D) biomimetic tumor model, comprising: employing a coaxial rotating cylinder system as disclosed herein and introducing a biomaterial into the empty annulus between the outer and inner cylinders; applying a phase-specific and force-guided polymerization of the biomaterial, wherein the phase specific polymerization comprises a nucleation phase and an elongation phase; thereby, producing a 3D biomimetic tumor model.

In such methods, suitably in the nucleation phase, a horizontal laminar Couette flow is generated by rotating the inner cylinder to promote the adsorption of the biomaterial monomers onto the inner cylinders surface to form an initial coating.

In such methods, suitably, in the elongation phase, a vertical gravitational force is applied for guiding the direction of biomaterial fibril assembly.

In certain embodiments, a multiple reaction or assessment may be conducted substantially simultaneously. For instance, in certain systems, a multiple-well plate or other multiple-reaction chamber system may be employed.

A variety of biomaterials may be utilized in the methods. For instance, in certain preferred embodiments, the biomaterial may comprise collagen I, collagen IV, Matrigel, poly L-lysine, Geltrex, gelatin, nitrogen, fibronectin, fibrinogen, gelatin methacrylate, fibrin, silk, pegylated gels, collagen methacrylate, decellularized extracellular matrices, basement membrane proteins, or any other biomaterial, or a combination thereof. In a preferred embodiment, the biomaterial is collagen I. Suitably in certain embodiments, the collagen I comprises a tumor cell, a spheroid, organoid and combinations thereof.

Suitably in certain embodiments, the tumor cell, spheroid or organoid and combinations thereof, are each surrounded by radially aligned fibers on one side and circumferentially oriented or aligned fibers the opposing side.

Suitably in certain embodiments, a higher fiber alignment is achieved than applying the gravitational force alone.

Suitably in certain embodiments, the coaxial rotating cylinders enable the seeding of tumor spheroids.

Suitably in certain embodiments, each tumor spheroid, organoid or cell is surrounded on one side by radially aligned fibers and the other side by circumferentially oriented or aligned fibers.

In another aspect, methods are provided for distinguishing tumors with different invasive and metastatic potentials, comprising, seeding a system or model as disclosed herein with different tumor cells and analyzing invasion patterns, border complexity and disseminated cell cluster number of each of the different tumor cells.

In another aspect, methods for diagnosing a neoplasia or cancer are provided, the methods comprising seeding a system or model as disclosed herein with cells from a subject's biological sample; and administering a chemotherapeutic agent to a subject diagnosed as having cancer.

In another aspect, methods are provided for screening for candidate therapeutic agents, comprising seeding a system or model as disclosed herein with tumor cells; adding a candidate therapeutic agent to determine effects, for example on cell death, invasion patterns, border complexity and/or disseminated cell cluster number of each of the different tumor cells. The candidate therapeutic agent suitably may be a compound known to have clinical use for cancer therapy, or a compound not yet established for cancer therapy use, or the candidate therapeutic agent may provide other activity that facilitates treatment.

Methods for treating a subject for cancer are also provided, wherein the methods may suitably comprise a) seeding a system or model as disclosed herein with one or more tumor cells; b) adding a candidate therapeutic agent to the tumor cells; c) determining effects of the candidate therapeutic agent on the tumor cells; and d) administering one or more selected candidate therapeutic agent to the subject. In one aspect, the tumor cells are obtained from the subject. In one aspect, multiple candidate therapeutic agents are assessed, including substantially simultaneously, for instance through use of a multi-well system or other multiple reaction chamber system. The one or more administered therapeutic agents may be selected from among multiple evaluated agents based on the determined effects of the candidate therapeutic agent on the tumor cells, for example a candidate therapeutic agent's effect on cell death, invasion patterns, border complexity and/or disseminated cell cluster number of the evaluated tumor cells.

Further provided is an assay for diagnosing a disease or affliction including cancer wherein the assay comprises assessing a tumor cell, a spheroid, or organoid and/or combinations thereof as disclosed herein.

Additionally provided is an assay for selecting one or more therapeutic agents to treat an identified patient, including a patient suffering from cancer wherein the assay comprises assessing a tumor cell, a spheroid, or organoid and/or combinations thereof as disclosed herein.

Further provided are assays to evaluate activity of one or more therapeutic agents between different patients in vitro to assess individual responses to the therapeutic agent(s) (e.g. anti-cancer agents) for patient-tailored personalized medicine purposes.

In certain aspects, the assay for use in personalized medicine is used to test individual patient responses to one or more therapeutic agents including where the disease of interest is a particular cancer. The assay may suitably include 1) treatment of one or more tumor cells, spheroids, and/or organoids (including where the tumor cells, spheroids, and/or organoids are derived from a patient of interest) with candidate therapeutic agents; and 2) analysis of the treated tumor cells, spheroids, and/or organoids e.g. by imaging or other assessment to determine the efficacy of the candidate therapeutic agent(s).

Further provided are assays and systems that include one or more tumor cells, spheroids, and/or organoids for assessment of the responsiveness to a particular treatment option, wherein the assessment comprises use of an assay or system as disclosed herein.

In certain aspects, the present systems and models are utilized or may be otherwise described as in vitro or ex vivo.

The article C. Su et al., Biomaterials 275 (2021) 120922 is included and incorporated herein as a portion of this disclosure.

Other aspects of the invention are disclosed infra.

### DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E shows alignment of collagen fibers by a phase-specific, force-guided method in a coaxial rotating cylinder system. (A) Proof of concept of a phase-specific, force-guided method. In the nucleation phase, a 2-minute horizontal laminar Couette flow is applied to adsorb collagen monomers on the cylinder glass surfaces. Then, in the elongation phase, the growth of collagen fibers is guided by a vertical gravitational force during a 20-minute gelling in a stationary condition. After an intact tube-shaped collagen gel is generated, the gel is cut and spread to form a large 3D collagen matrix with aligned fibers. A computer-aided design (B) and device images (C) of coaxial rotating cylinder-system. The system consists of two glass cylinders concentrically aligned by a 3D printed base, a brass rod, and plastic bearings. Collagen is polymerized in the space between the two coaxial cylinders (arrow). (D) Coaxial rotating cylinder system is powered by a motor and speed controller system. (E) A tube-shaped collagen gel is taken out from the coaxial rotating cylinder system. After the tube-shaped collagen gel is cut and spread, a large collagen matrix with aligned fibers is generated.

FIGS. 2A-2D shows computational fluid dynamic simulation of laminar Couette flow in collagen solution. (A) A 3D plot of the fluid velocity field exhibits a laminar Couette flow in collagen solution with the inner cylinder rotating at 50 rpm. (B) 2D plots of the fluid velocity field demonstrates a laminar Couette flow with the inner cylinder rotating at 50 rpm and 500 rpm, respectively. (C and D) The desired shear rate (C) of 35 s⁻¹ and flow velocity (D) of 0.03 m/s are identified for the inner cylinder rotation at 50 rpm to align collagen fibers.

FIGS. 3A-3G show the laminar Couette flow followed by gravitational force enhances fiber alignment. Images of multiphoton second-harmonic generation (A) and computational segments (B) of collagen fibers in random (fiber polymerization on a glass slide), gravity only (fiber polymerization by gravitational force only), and Couette+gravity groups (fiber polymerization by laminar Couette flow with subsequent gravitational force). (C) The angular frequency distribution of collagen fibers within one gel for each experimental group (n=20 images). Red lines represent the magnitude and direction of mean resultant vectors. (D) A directional statistics analysis reveals a more aligned but slightly deviated collagen fiber orientation in a Couette+gravity sample compared to a gravity only sample within one gel for each experimental group (n=20 images). ***p<0.001, ****p<0.0001 by Watson-Williams test (E) Scanning electron microscope images of collagen fibers exhibit more aligned but oblique-oriented collagen fibers in a Couette+gravity group compared to a gravity only group. (F) Frequency distribution of collagen fiber orientation in random, gravity only, and Couette+gravity groups. Data are mean±SEM (20 images per gel from three gels for each experimental group). (G) The resultant vector length of collagen fibers, representing fiber alignment, is greatest in Couette+gravity, followed by gravity only and random groups. Data are mean and 95% confidence interval (CI) (20 images per gel from three gels for each experimental group). *p<0.05, **p<0.01, ***p<0.001 by one-way ANOVA.

FIGS. 4A-E show couette flow with subsequent gravitational force builds a dual topographical tumor spheroid model. (A) Proof of concept schematic for dual topographical tumor spheroid model. (B) Multiphoton second-harmonic generation and computational segmented images of collagen fibers on day 0 after collagen polymerization with embedded tumor spheroids. (C) Frequency distribution and (D) alignment (resultant vector length) of collagen fiber orientation showing collagen fibers were more aligned in radial zone of tumor spheroids in Couette+gravity group (fiber polymerization by laminar Couette flow with subsequent gravitational force). (E) Fiber density analysis showed no difference in fiber density between circumferential and radial zone in all experimental conditions. (F) Orthogonal view of a T47D spheroid on day 0 in Couette+gravity group. (C) Data are mean±SEM (n=15 spheroids per group from three independent experiments). **p<0.01, ***p<0.001, ****p<0.0001 by Kolmogorov-Smirnov test for comparing frequency distributions. (D-E) Data are mean and 95% CI (n=15 spheroids per group from three independent experiments). *p<0.05, **p<0.01, ***p<0.001, ****p<0.0001 by one-way ANOVA.

FIGS. 5A-F show dual topographical tumor spheroid model reveals cancer invasion pattern determined by matrix topography. (A) MCF7 spheroids in dual topographical model (fiber polymerization by laminar Couette flow with the subsequent gravitational force) after a 10-day invasion manifests evenly distributed spheroids with the same invasion pattern along radially aligned fibers. (B) Multiphoton second-harmonic generation images of T47D tumor spheroids after a 10-day invasion show no collagen fiber orientation changes. Confocal images of (C) T47D and (D) MCF7 tumor spheroids after a 10-day invasion exhibit multicellular disseminated clusters and finger-like projections invading along radially aligned fibers with a magnified inset for multicellular disseminated cell clusters. (E) MCF7 and (F) T47D tumor spheroids in the Couette+gravity group display more complicated borders than random and gravity only groups. (E-F) Box and whisker plot with mean and 95% CI (n=20 spheroids per group from three independent experiments). ****p<0.0001 by one-way ANOVA.

FIGS. 6A-E shows radially aligned fiber topography promotes cell cluster-based collective cancer invasion. (A) A binary image of an MCF7 tumor spheroid after a 10-day invasion. (B) Disseminated cell clusters invade along the direction of radially aligned or circumferentially oriented or aligned fibers. Red lines represent the magnitude and direction of mean resultant vectors. (C) More clusters of cells are disseminated from main tumors on the radial zone than circumferential zone. **p<0.01, ***p<0.001 by Student's t-test (D) Border complexities are greater on the radial zone than circumferential zone. ****p<0.0001 by Wilcoxon matched-pairs signed-rank test (E) Cell numbers of the disseminated cell clusters are higher on the radial zone than circumferential zone. *p<0.05, ***p<0.001 by Student's t-test (C and E) Box and whisker plot with mean and 95% CI (C-E) n=20 spheroids per group from three independent experiments.

FIGS. 7A-G show sual topographical tumor model distinguishes tumor spheroids and organoids invasion pattern. (A) Time-lapse images of MMTV-PyMT and C3(1)-Tag mouse mammary tumor organoids in dual topographical tumor models. (B) Multiphoton second-harmonic generation and confocal images of T47D and MDAMB231 human breast tumor spheroids and MMTV-PyMT and C3(1)-Tag mouse mammary tumor organoids after a 4-day invasion. (C) The fiber orientation frequency distribution of originally radial and circumferential zones after a 4-day invasion indicates fiber orientation remodeling by MDAMB231 spheroids and C3(1)-Tag organoids but neither by T47D spheroids nor MMTV-PyMT organoids. Data are mean±SEM (n=20 spheroids or organoids per group from three independent experiments). (D) Border complexity, (E) disseminated cell cluster number, and (F) disseminated cell cluster size of tumor spheroids/organoids. (G) In vivo invasiveness, molecular subtypes, and in vitro invasion pattern in dual topographical model of tumor spheroids/organoids. (D-F) Box and whisker plot with mean and 95% CI (n=20 spheroids/organoids per group from three independent experiments). *p<0.05, **p<0.01, ***p<0.001, ****p<0.0001 by one-way ANOVA.

FIGS. 8A-C, FIGS. 9A-R and FIG. 10 show further results of the examples which follow.

FIG. 11 (includes FIGS. 11A-G) shows in FIG. 11A: a set of coaxial cylinders in a multiwell system. In a multiwell system, each well in a multiwell plate serves as the outer cylinder and a plastic tube as the inner cylinder. The spinning of the inner plastic tube aligns the collagen fibers by a phase-specific, force-guided method. FIGS. 11B-11C: A computer-aided design of (B) a motor holder and (C) a connector for the motor shaft of a coaxial cylinder system. FIG. 11D: A representative image of a multiwell coaxial cylinder system consists of a 3D printed motor holder, a motor, a connector for the motor shaft, a plastic tube as the inner cylinder, and a 24 well plate. The inner and outer cylinders are concentrically aligned by the motor holder and the connector. FIG. 11E: A multiphoton second-harmonic generation image shows aligned collagen fibers generated by a 24-well multiwell coaxial cylinder system. FIG. 11F: The angular frequency distribution of collagen fibers demonstrates highly aligned collagen fibers with a peak orientation around 90°. Red line represents the magnitude and direction of mean resultant vectors. FIG. 11G: A computer-aided design of motor holder designed for a 96-well coaxial cylinder system.

FIG. 12 shows results of Example 6 which follows.

### DETAILED DESCRIPTION

As used herein, a tumor organoid or spheroid means a cell mass containing aggregates of tumor cells. A tumor organoid or spheroid may be of a variety of sizes and may include for example 50 or 100 to 10000 or more cells and may have the longest dimension of from example 0.1 mm to 1 or 2 mm, more typically example 0.1 mm to 1 mm. The tumor may be for example colon cancer, gastric cancer, prostate cancer, breast cancer, cervical cancer, ovarian cancer, bladder cancer, lung cancer, hepatocellular carcinoma, kidney cancer, or pancreatic cancer, or other. The term organoid is understood to embrace spheroids.

The term organoid as used herein may refer to a collection of organ specific cell mass that develop from stem cells or tumor initiating cells and self-organizes similar to in vivo. See Lancaster, Science 345(6194), 2014: 1247125.

In certain aspects, the term “organoid” refers to an in vitro collection of cells which resemble their in vivo Counterparts and form 3D structures.

The term “aligned” or “oriented” refers to the orientation of a matrix material wherein at least about 55 or 60% of the fibrous structures or materials are oriented in a defined direction and their orientation forms either a single axis or multiple axes of alignment. More preferably, at least about 70, 80, 85 or 90% of the fibrous structures or materials are oriented in a defined direction. The orientation of any given fiber can deviate from a given axis of alignment and the deviation can be expressed as the angle formed between the alignment axis and orientation of the fiber. A deviation angle of 0° exhibits perfect alignment with the given axis and 900 (or −90°) exhibits orthogonal alignment of the fiber with respect to the given axis of alignment. When multiple axes of alignment exist in a given layer, the alignment of a particular fiber is determined in relation to its closest axis. In exemplary embodiments, the standard deviation of the aligned fibers from their closest axes of alignment can be an angle selected from between 0° and 10, between 0° and 30, between 0° and 5°, between 0° and 10°, between 0° and 20°, or between 0° and 25°.

The term “phase-specific, force-guided method” refers to a method to align matrix fibers (e.g. collagen fibers) having differential alignment regions (particularly a first region of radially aligned fibers and a second regions of circumferentially aligned fibers) by applying two different forces designed for the two phases of fiber polymerization, nucleation and elongation phases. The two different forces may include 1) laminar flow or force (e.g. horizontal laminar Couette flow) in nucleation phase and 2) gravitational force in elongation phase. In a preferred procedure, first monomers to form the matrix material (e.g. collagen monomers) adsorb on the surface in the nucleation phase, then followed by monomers growing into fibers in the elongation phase. The phase-specific, force-guided method is designed based on the two-phase nature of fiber polymerization. During the nucleation phase, a horizontal laminar Couette flow driven by inner cylinder rotation of the disclosed system deposits matrix material monomers on the cylinder surfaces (e.g. glass surfaces). Then, in the elongation phase, the stop of inner cylinder rotation changes the force orientation to vertical gravitational force to guide fiber growth. The Examples which follow also exemplify preferred phase-specific, force-guided methods to align.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

As used in the description herein and throughout the claims that follow, the meaning of “a,” “an,” and “the” includes plural reference unless the context clearly dictates otherwise.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

### Cells/Organoids

A variety of tumor cells may be utilized in the present systems including tumor cells of a carcinoma, a sarcoma, a lymphoma, or other. Cells derived from a carcinoma can include for example cells derived from an adenocarcinoma and/or a squamous cell carcinoma. Cells from a sarcoma may include for example cells from an osteosarcoma, a chondrosarcoma, a leiomyosarcoma, a rhabdomyosarcoma, a fibrosarcoma, an angiosarcoma or other. Cells from a lymphoma may include for example cells derived from a Hodgkin lymphoma, a non-Hodgkin lymphoma, or a combination thereof.

As discussed, the cells may be derived or obtained from a particular subject or human patient that has suspected cancer.

Organoids can be produced by known methods, including using tumor cells as disclosed herein, for example tumor cells obtained for an identified human patient and which obtained cells may be cultured. See exemplary methods disclosed in C. Su et al., Biomaterials 275 (2021) 120922, V. Padmanaban et al., Nat Protoc 15(8) (2020) 2413-2442. See also methods disclosed in US2022/0081679.

Cells or tissue organoids derived from kidney, heart, liver, brain, lung or stomach can be readily obtained. For instance, such cells may be obtained from as biopsy from a mammal, including a human. The cells also may be stem-cell derived. Suitable kidney, heart, liver, brain, lung or stomach also may be commercially available.

Tissue organoids derived from kidney, heart, liver, brain, lung or stomach can be produced by known methods, including using cells obtained from biopsy from a subject, and which obtained cells may be cultured. See exemplary methods disclosed in Sato, T. et al. Single Lgr5 stem cells build crypt-villus structures in vitro without a mesenchymal niche. Nature 459, 262-265 (2009). https://doi.org/10.1038/nature07935 See also C. Su et al., Biomaterials 275 (2021) 120922, V. Padmanaban et al., Nat Protoc 15(8) (2020) 2413-2442, and the methods disclosed in US2022/0081679.

### Matrix

A variety of matrix materials may be use in the present systems including for example a collagen, an elastin, a fibronectin, or a combination thereof. Preferred matrix materials may be present in vivo or in ex vivo association with tumor cells, or with kidney, heart, liver, brain, lung or stomach cells.

Preferred matrix materials may comprise a collagen. A variety of collagen may be utilized of example collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, or a combination thereof. In addition to above biological matrices, synthetic matrix materials may be used.

Prior to cancer cell invasion, the structure of the extracellular matrix (ECM) surrounding the tumor is remodeled, such that circumferentially oriented or aligned matrix fibers become radially aligned. This predisposed radially aligned matrix structure serves as a critical regulator of cancer invasion. However, a biomimetic 3D model that recapitulates a tumor's behavioral response to these ECM structures is not yet available. In this study, we have developed a phase-specific, force-guided method to establish a 3D dual topographical tumor model in which each tumor spheroid/organoid is surrounded by radially aligned collagen I fibers on one side and circumferentially oriented or aligned fibers on the opposite side. A coaxial rotating cylinder system was employed to construct the dual fiber topography and to pre-seed tumor spheroids/organoids within a single device. This system enables the application of different force mechanisms in the nucleation and elongation phases of collagen fiber polymerization to guide fiber alignment. In the nucleation phase, fiber alignment is significantly enhanced by a horizontal laminar Couette flow driven by the inner cylinder rotation. In the elongation phase, fiber growth is guided by a vertical gravitational force to form a large collagen matrix gel (35×25×0.5 mm) embedded with >1,000 tumor spheroids. The fibers above each tumor spheroid are radially aligned along the direction of gravitational force in contrast to the circumferentially oriented or aligned fibers beneath each tumor spheroid/organoid, where the presence of the tumor interferes with the gravity-induced fiber alignment. After ten days of invasion, there are more disseminated multicellular clusters on the radially aligned side, compared to the side of the tumor spheroid/organoid facing circumferentially oriented or aligned fibers. These results indicate that our 3D dual topographical model recapitulates the preference of tumors to invade and disseminate along radially aligned fibers. We anticipate that this 3D dual topographical model will have broad utility to those studying collective tumor invasion and that it has the potential to identify cancer invasion-targeted therapeutic agents.

Cancer progression is a dynamic process of tumor cells interacting with their microenvironment [1]. Cancer cells interact with tumor stromal cells to continuously remodel their microenvironment even before local invasion [2, 3] and distant metastasis [4, 5]. In turn, the remodeled tumor microenvironment distinguishes itself from normal tissue by providing biophysical and biochemical cues as a route of cancer invasion [6, 7]. Together, the reciprocal interaction between cells and extracellular matrix (ECM) forms a synergistic loop to drive tumor progression. Structural remodeling of the ECM surrounding tumors is one consequence of cell-ECM interaction [8]. Invading cancer cells align surrounding ECM fibers to form a “migration highway,” which guides tumor cells to efficiently penetrate through stroma [9-11]. Furthermore, the predisposed tumor ECM structure at the tumor border can be formed even before cancer invasion. For example, the alteration of the stromal microenvironment is a major factor driving the progression from preinvasive breast cancer, ductal carcinoma in situ (DCIS), to invasive ductal carcinoma (IDC). In contrast, there are only modest genetic changes between cancer cells in IDC and DCIS [2, 12]. In DCIS patients, the predisposed radially oriented matrix structure predicts poor prognosis [13]. Direct evidence from experimental mouse models reveals that radially aligned fibrillar collagen structure promotes breast tumor invasion [14, 15]. Also, an in vitro tumor model demonstrates that tumor spheroids can remotely orient collagen fibers up to a distance of five times the spheroid radius from the spheroid border [16].

Regarding the ECM-to-cell effect, previous studies indicated that aligned fiber topography guides tumor cell movement by enhancing migration persistence and velocity [9, 17]. However, most studies focused on responses of individual cells to aligned topography, and little is known about how tumor spheroids or organoids react to predisposed ECM structure, mainly due to the lack of relevant experimental models [18-21]. Unlike matrices containing scattered individual cells, tumor spheroid models better recapitulate in vivo collective cell migration, tumor invasion, and metastasis [22]. However, elucidating how tumor spheroids/organoids respond to various ECM structures requires more complicated bioengineering approaches. A major obstacle in engineering a 3D topographical tumor spheroid/organoid model is to encompass multicellular tumor spheroids with specialized ECM architecture without damaging ECM structure.

Previous studies created aligned collagen fibers by applying different methods such as extensional strain [23-25], electrospinning [26-28], magnetic field [29, 30], microfluidics [31-33], and cell remodeling [18, 19, 34]. Among all the existing methods to align ECM fibers, cell remodeling [18, 19, 34] and microfluidics [20, 21, 25] are the most commonly used to build tumor models with a predisposed ECM structure (see Table 1 below). However, common limitations of these methods are that fiber alignment is confined to a small or restricted area [20, 34] and difficulties with embedding tumor spheroids in the matrix. Thus, embedding or attaching tumor spheroids to the matrix after the formation of fiber alignment risks interrupting the preformed ECM structure [18, 19, 34]. Microfluidic methods may only be suitable for single-cell models and are difficult to apply to tumor spheroid models because the pre-seeded tumor spheroids interfere with the flow that drives fiber alignment. In addition, it is difficult to embed tumor spheroids in electrospun scaffolds due to their low porosity [35]. Therefore, despite their utility for studying individual cell invasion, currently available models of aligning matrix fibers are not applicable to study collective cell invasion of tumor spheroids/organoids. A novel model that enables us to seed tumor spheroids/organoids and control fiber pre-alignment is needed to investigate collective cancer cell invasion.

To develop a 3D topographical tumor spheroid/organoid model, we established a novel method to efficiently create a large-scale collagen gel with tumor spheroids/organoids surrounded by dual ECM topography. We applied a phase-specific, force-guided method for collagen polymerization. [32, 36]. In the nucleation phase, the first phase of collagen polymerization, a horizontal laminar Couette flow was generated by rotating the inner cylinder in a coaxial rotating cylinder system to promote the adsorption of collagen monomers onto the surface and form an initial coating of collagen. Next, in the elongation phase, a vertical gravitational force was adopted to guide the direction of collagen fibril assembly. This new topography system presents several advantages. First, a higher fiber alignment is achieved than applying the gravitational force alone. Second, unlike most microfluidics, our coaxial rotating cylinders enable the seeding of tumor spheroids. Third, each tumor spheroid is surrounded on one half by radially aligned fibers and the other half by circumferentially oriented or aligned fibers. Since the individual tumor spheroids are interacting simultaneously with the two most common topographical features of tumor stroma, our model is ideal for studying how 3D topography affects tumor invasion. Our results indicate that radially aligned topography promotes tumor invasion by enhancing cluster-based dissemination of tumor cells. Disseminated multicellular clusters budding out from the main tumor on radially aligned collagen fibers in our 3D model authentically recapitulates human cancer invasion. We anticipate that our 3D topographical tumor model can be readily applied to investigate collective invasion across cancer types and to identify new cancer therapies.

The tumor cells, spheroids, and/or organoids as disclosed herein can be used to test libraries of chemicals (including small molecules), antibodies, natural products or other agents for suitability for use as drugs or preventative medicines. The candidate therapeutic agents can be new or modified drugs and compounds.

In certain aspects, cells or tissues from a patient of interest, such as tumor cells from the patient, can be cultured and then treated with a drug or a screening library. It is then possible to determine the effectiveness of the candidate agent against the tumor cells, spheroids, and/or organoids. This allows specific patient responsiveness to a particular drug to be tested, thus allowing treatment to be tailored to a specific patient.

In certain aspects, the assay as disclosed herein comprising the tumor cells, spheroids, and/or organoids is a drug screen, where the tumor cells, spheroids, and/or organoids are derived from one individual patient. In certain aspects, the tumor cells, spheroids, and/or organoids in a drug screen, for example in an array, are derived from different patients.

Libraries of molecules can be used to identify a molecule that affects the tumor cells, spheroids, and/or organoids. In certain aspects, libraries comprise antibody fragment libraries, peptide phage display libraries, peptide libraries, lipid libraries, small molecule compound libraries, or natural compound libraries (e.g. Specs, TimTec). Additionally, genetic libraries can be used that induce or repress the expression of one or more genes in the progeny of the stem cells. These genetic libraries comprise cDNA libraries, antisense libraries, and siRNA or other non-coding RNA libraries. The tumor cells, spheroids, and/or organoids can be exposed to multiple concentrations of a test agent for a certain period of time. At the end of the exposure period, the cultures are evaluated. The term “affecting” is used to cover any change in a cell, including, but not limited to, a reduction in, or loss of, proliferation, a morphological change, and cell death.

In certain aspects, the present systems and assays include tumor cells, spheroids, and/or organoids that are patient derived and comprise treatment of such tumor cells, spheroids, and/or organoids with one or more candidate therapeutic agents, for example for use in personalized medicine, e.g., to test individual patient response to the candidate therapeutic agent for a disease of interest, particularly cancer. The candidate therapeutic agents may be anti-cancer agents.

As discussed, a plurality of assays as disclosed herein may be run in parallel such as using a multi-well reaction plate. Such assays maybe run for example with different therapeutic agents, or different concentrations of a particular therapeutic agent to obtain a differential response to the various concentrations. Effective concentration of an agent can be assessed using a range of concentrations resulting from 1:10, or other log scale, dilutions. The concentrations may be further refined with a second series of dilutions, if necessary.

### EXAMPLES

### Example 1

### Materials and Methods

### 1. Design and Assembly of the Coaxial Rotating Cylinder System

Our coaxial rotating cylinder system comprises two borosilicate glass scintillation vials (Sigma-Aldrich, USA) with different radii that were concentrically aligned by a customized 3D printed base, a brass rod, and plastic bearings. The outer cylinder is held fixed by the base, while the inner cylinder is free to rotate about its axis. The portion above the neck of the outer glass vial was cut off by a glass cutter for the inner glass vial to fit in. The rotation of the inner glass cylinder was powered by a direct current 6 volt 500 revolutions per minute (rpm) micro speed reduction motor, and the rotation speed was controlled by a pulse-width modulation stepless direct current motor speed controller. The brass rod attached to the inner glass cylinder was connected to the motor shaft by a customized 3D-printed part. The 3D printed base and parts were designed using Autodesk Inventor software (Autodesk, USA) and printed by a desktop 3D printer (Cubicon, Korea) with acrylonitrile butadiene styrene filaments. After being concentrically aligned, the smaller inner glass cylinder was placed inside the center of the larger outer glass cylinder leaving an empty annulus between the two cylinders for collagen gelling.

2. Fabrication of Collagen Matrices with Aligned Fibers

Collagen matrices were prepared by mixing type I rat tail telocollagen solution and neutralization solution in a ratio of 9:1 at a final concentration of 3.69 mg/ml (lot. 8282, RatCol® Rat Tail Collagen for 3D Hydrogels, Advanced BioMatrix, USA). Type I collagen solution was kept on ice before mixing. After mixing 900 μL of type I collagen solution with 100 μL neutralization buffer, a 1 mL collagen pregel solution was poured into the space between the two glass cylinders, and the inner cylinder was immediately rotated. A 2-minute rotation of the inner cylinder at 50 rpm was applied to generate Couette flow for collagen monomer nucleation on the glass surface and followed by a 20-minute gelling in a stationary condition for the gravitational force to guide collagen fiber elongation. Collagen was polymerized at room temperature.

### 3. Computational Fluid Dynamic Simulation

A computational fluid dynamic simulation was performed using COMSOL Multiphysics version 5.5 (COMSOL, USA). First, a 2D geometry of a rectangle with the cross-section dimensions of the space between two glass cylinders was built. The density and dynamic viscosity of the collagen solution were input as material properties. Then, to simulate the fluid dynamics with the inner cylinder rotating, the inner wall of the 2D rectangle was set as a sliding wall, and the center axis of both cylinders was fixed as the symmetry. Laminar flow was applied as the physical model, and the fluid flow was described following Navier-Stokes equations [37]. Finally, the parameter sweep was set under various rotation speeds of the inner cylinder to determine the ideal shear rate for collagen nucleation.

### 4. Alignment and Orientation Analysis of Collagen Fibers

The collagen fibers in gels were visualized by an Olympus FV1000 multiphoton second-harmonic generation (SHG) microscope (Olympus, Japan) or a multiphoton second-harmonic generation (SHG) and confocal microscope (Zeiss LSM 710NLO-Meta, Germany). Images of 20 randomly picked locations were taken for each collagen gel. The SHG microscopic images were segmented and analyzed computationally by CT-FIRE, a MATLAB-based program, to quantify the orientation of collagen fibers [38]. To compare the alignment and orientation between experimental conditions, we performed directional statistics analysis using CircStat, a MATLAB program for circular statistics [39].

The fiber alignment was determined by resultant vector length from 20 random images for each gel. The value of resultant vector length ranges between 0 and 1. When the value is closer to 1, the fiber orientation angle is more concentrated around the mean direction, indicating more aligned fibers.

$$Resultant vector length = \sqrt{{( \frac{1}{N} {\sum }_{i = 1}^{N} cos {θ}_{i} )}^{2} + {( \frac{1}{N} {\sum }_{i = 1}^{N} sin {θ}_{i} )}^{2}}$$

An alignment index representing the peakedness was introduced as a second method to assess the fiber alignment. The alignment index is equal to the highest frequency percentage (h) of angular distribution divided by the half of full width at a half maximum (FWHM) [24]. A value of 0 represents random distribution. The higher the value is, the more aligned the fibers are.

$$Alignment index = \frac{h}{FWHM \times 1 / 2}$$

The fiber orientation was represented by mean resultant vector. The mean resultant vector between experimental conditions was tested by the Watson-Williams test [39].

$$Mean resultant vector = \frac{1}{N} {\sum }_{i = 1}^{N} ( \begin{matrix}cos {θ}_{i} \\ sin {θ}_{i}\end{matrix} )$$

### 5. Scanning Electron Microscopy (SEM)

Images of the fibrous collagen morphology were taken using SEM (Apreo, Thermo Fisher Scientific, USA). The collagen gel samples were lyophilized at −80° C. and high-vacuum status (0.07 millibar) via a Freeze Dry System (FreeZone Plus 2.5 Liter Cascade Benchtop Freeze Dry System, Labconco, USA). The lyophilized samples were coated with Au/Pt by a sputter coater for 60 seconds. Next, the samples were placed into the SEM vacuum chamber, and a 5 kV of accelerating voltage was applied to acquire high-resolution images.

### 6 Cell Culture

MCF7, T47D, and MDAMB231 human breast cancer cells were purchased from American Type Culture Collection (VA, USA). MCF7 and MDAMB231 cells were maintained in Dulbecco's Modified Eagle's Medium (DMEM) (Gibco, USA), and T47D cells were maintained in RPMI 1640 medium (Gibco, USA). Media were supplemented with 10% fetal bovine serum (Thermo Fisher Scientific, USA) and 1% penicillin-streptomycin (10,000 U/mL) (Thermo Fisher Scientific, USA). The cells were incubated under a 5% CO₂ humidified atmosphere at 37° C.

### 7. Development of Dual Topographical Tumor Spheroid and Organoid Model

Tumor spheroids were generated using AggreWell 400 6-well microwell culture plates (STEMCELL Technologies, USA) [40]. Before seeding cells in microwell culture plates, 0.5 mL anti-adherence rinsing solution (STEMCELL Technologies, USA) was added into each well, and a 2-minute 2000 g centrifugation followed by a 30-minute incubation at 37° C. was performed to prevent cell adhesion onto the microwells. Next, a 2.5 million single-cell suspension in 2 mL was seeded in each well. A 5-minute 200 g centrifugation was performed to cluster the cells in microwells, and the cells were incubated in a CO₂ incubator at 37° C. overnight for cells to aggregate and form spheroids. Tumor spheroids generated from a well of a 6-well microwell culture plate were harvested, and one-fourth of the tumor spheroids in 100 μL medium were mixed and seeded together within a 1 mL collagen pregel solution (900 μL of type I collagen solution and 100 μL neutralization buffer) at a final concentration of 3.35 mg/ml (lot. 8282, RatCol® Rat Tail Collagen for 3D Hydrogels, Advanced BioMatrix, USA), and poured into the space between the two coaxial cylinders.

Mouse mammary tumor organoids were derived from two genetically engineered mouse models of breast cancer, MMTV-PyMT [41] and C3(1)-Tag [42], as described previously [43]. Mammary tumors harvested from MMTV-PyMT or C3(1)-Tag mice were mechanically minced and enzymatically digested by collagenase and trypsin. Single cancer cells or stromal cells were separated from epithelial tumor organoids by a series of differential centrifugation. Around 1500 mammary tumor organoids in 100 μL of medium were mixed with a 1 mL collagen pregel solution and seeded together into the space between the two coaxial cylinders. All mice were female and were obtained from The Jackson Laboratory (Bar Harbor, ME). All procedures were conducted by following protocols approved by the Johns Hopkins Medical Institute Animal Care and Use Committee (IACUC).

For the Couette+gravity group, collagen fibers were polymerized in the coaxial cylinder system under a 2-minute laminar Couette flow driven by inner cylinder rotation at 50 rpm followed by a 20-minute gravity-driven fiber elongation. For the gravity only group, collagen fibers were polymerized in the coaxial cylinder system with a 20-minute gelling in a stationary condition. Collagen was polymerized at room temperature. The resulting tube-shaped collagen gels embedded with tumor spheroids/organoids were then cut and spread out to form dual topographical tumor models. MCF7 and MDAMB231 tumor spheroids were maintained in DMEM medium (Gibco, USA), and T47D tumor spheroids were maintained in RPMI 1640 medium (Gibco, USA). Media were supplemented with 10% fetal bovine serum (Thermo Fisher Scientific, USA) and 1% penicillin-streptomycin (Thermo Fisher Scientific, USA). MMTV-PyMT and C3(1)-Tag tumor organoids were maintained in DMEM-F12 medium (Gibco, USA) supplemented with 1% insulin-transferrin-selenium (Gibco, USA), 1% penicillin-streptomycin (Sigma, USA), and 2.4 nM FGF2 (Sigma, USA).

### 8. Cancer Invasion Pattern Analysis

After a 10-day culture in collagen gels, tumor spheroids were stained with CellTracker Red CMTPX Dye (Thermo Fisher Scientific, USA) and Hoechst 33342 (Thermo Fisher Scientific, USA) and fixed with 4% paraformaldehyde (Thermo Fisher Scientific, USA). A spinning disk confocal microscope (Nikon Tie inverted widefield microscope and Yokogawa WI spinning disk, Japan) or a multiphoton second-harmonic generation (SHG) and confocal microscope (Zeiss LSM 710NLO-Meta, Germany) was used to image tumor spheroids. Images were analyzed by a customized macro in Image J. In brief, Z stack confocal images of a whole tumor spheroid/organoid were processed by Background Subtraction, Z projection, and Make Binary. The area, perimeter, orientation angle, and other parameters of binary images were quantified with the Analyze Particles function. Disseminated cell clusters were defined as cells with no continuous connection with the main tumor in binary images. The morphology complexity of tumor spheroids was presented by border complexity [44].

$$Border complexity = \frac{{( \sum peri m e t e r )}^{2}}{4 π \times \sum area}$$

The higher the border complexity, the more irregular the tumor. When invasion occurs, the invasion projection increases the border complexity by a greater border perimeter for a corresponding are A. When disseminated cell clusters appear, the perimeter and area of both the main spheroid and disseminated cell clusters were taken into calculation. To compare the border complexity between radial and circumferential zone in the same tumor spheroids/organoids, we divided the binary image of a whole tumor spheroid/organoid into two images using the widest short axis of the spheroid/organoid as the separating line.

### 9. Statistical Analysis

Data were analyzed using GraphPad Prism 9 (GraphPad Software, USA). The frequency distributions collagen fiber orientation was compared by Kolmogorov-Smirnov test Fiber alignment (resultant vector length) between experimental conditions was compared by one-way ANOVA. The border complexity of tumor spheroids/organoids and the number and size of disseminated cell clusters between experimental groups were compared by one-way ANOVA. Spheroid morphology in different ECM structures was compared using the Student's t-test, one-way ANOVA, or Wilcoxon matched-pairs signed-rank test For directional statistics, the alignment and orientation parameters of collagen fibers were analyzed on a MATLAB program, CircStat [39]. The mean resultant vectors between samples were compared by the Watson-Williams test For all statistical analyses, the difference was considered significant at p<0.05.

### Results

### 1. Collagen Fibers are Aligned Using a Phase-Specific, Force-Guided Method

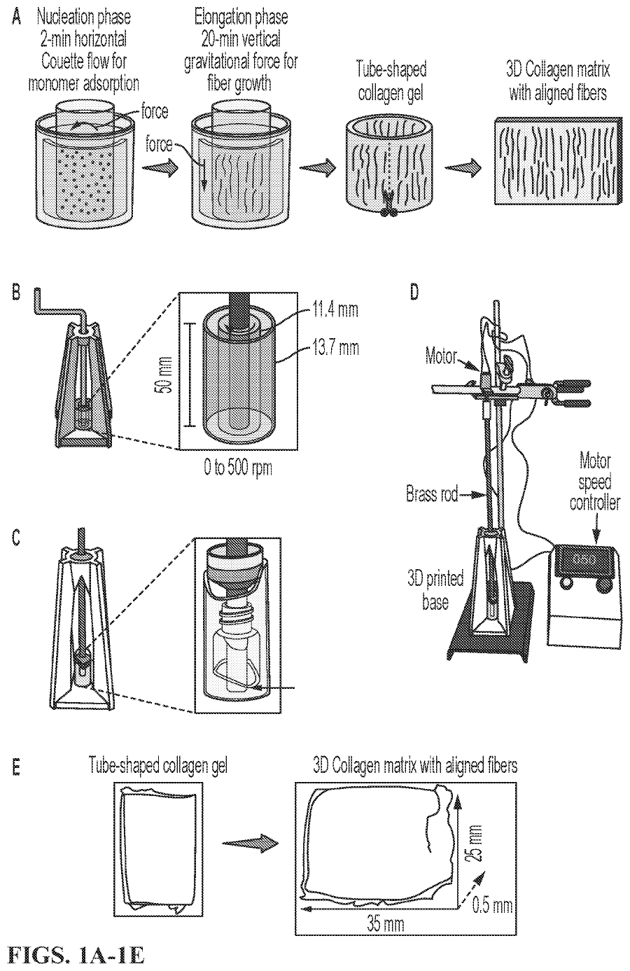

A proof of concept to develop a phase-specific, force-guided method for aligning collagen fibers in a 3D matrix gel is based on the two-phase nature of collagen fiber polymerization, with nucleation and elongation phases [32, 36]. Type I collagen was used to create the 3D matrix for modeling breast cancer invasion because it is one of the most abundant ECM components in breast tumors and plays a critical role in tumor progression [45, 46]. FIG. 1A shows the working principle and fabrication process of our method to create a large collagen gel with aligned fibers. During the nucleation phase, we applied a horizontal laminar Couette flow to deposit collagen monomers on the cylinder glass surface. This increases the initial collagen monomer coating, which forms a collagen mat on the glass substrate, improving the collagen fiber formation and alignment [32]. The nucleation of collagen occurred within the first two minutes of polymerization [32]. Therefore, the inner cylinder was set to rotate for the first 2 minutes to adsorb a collagen monomer mat onto the surface to form an initial coating of collagen. Then, after inner cylinder rotation was stopped, the force orientation was changed to vertical gravitational force to guide collagen fibers to grow vertically in the elongation phase [31, 32].

### 2. A Coaxial Rotating Cylinder System is Applied to Align Collagen Fibers in a 3D Matrix

To develop a device that accommodates forces for both the nucleation and elongation phases, we applied a coaxial rotating cylinder instead of microfluidics for the following reasons (FIG. 1A). First, as a closed system, the shear force driven by laminar Couette flow can be easily controlled by tuning the rotation speed of the cylinder without external equipment such as a syringe pump. Since the nucleation phase occurs quickly within two minutes [32], our system does not need a syringe pump to drive the flow, thereby shortening the preparation time and reducing the probability of nucleation occurring outside of the system. Second, stopping the inner cylinder rotation immediately shifted the laminar shear flow to the vertical gravitational force to allow collagen fiber elongation. Third, our method creates a large collagen gel with homogeneously aligned fibers compared to the limited space in microfluidics. Fourth, our coaxial rotating cylinders system is a stable environment to seed spheroids/organoids inside collagen during polymerization [47].

We designed our coaxial rotating cylinder system to create a laminar Couette flow upon rotation of the inner cylinder. We used a larger borosilicate glass scintillation vial (radius of 13.7 mm) as the outer cylinder and a smaller borosilicate glass scintillation vial (radius of 11.4 mm) as the inner cylinder (FIG. 1B). The radius ratio of the two concentric cylinders was 0.83, which is >0.8, the criteria to form laminar Couette flow [47]. To coaxially align the inner and outer cylinders, we designed and 3D-printed a pyramid-shaped base, together with a brass rod and plastic bearings to hold the inner cylinder in the middle of the outer cylinder. (FIG. 1C). The resulting open-topped annulus between the inner and outer cylinders allowed space to pour the mixture of collagen solution and neutralization buffer. Then, the laminar Couette flow driven by the rotating inner cylinder initiated the coating of collagen monomers on the cylinder glass surface. The rotation speed ranging from 0 to 500 rpm was stably regulated by a motor and stepless motor speed controller (FIG. 1D).

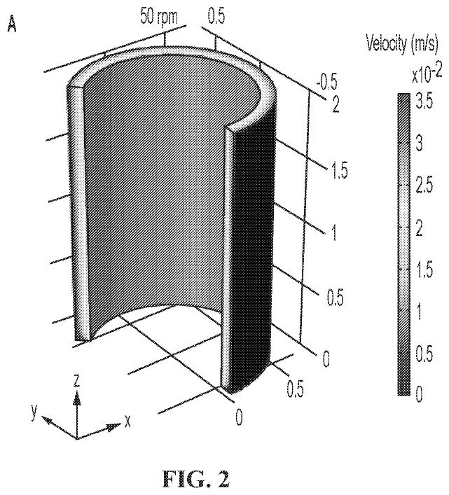

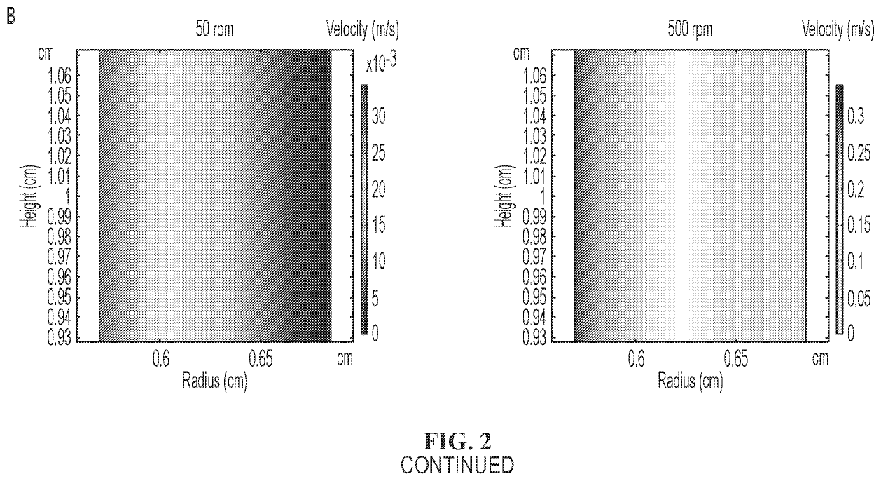

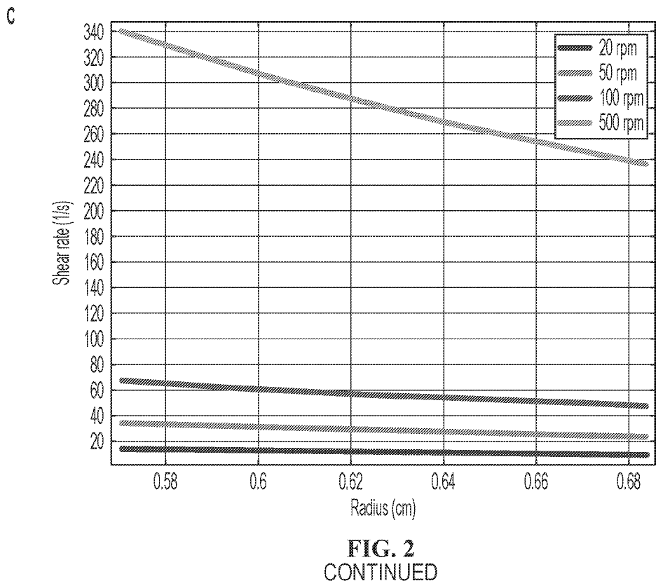

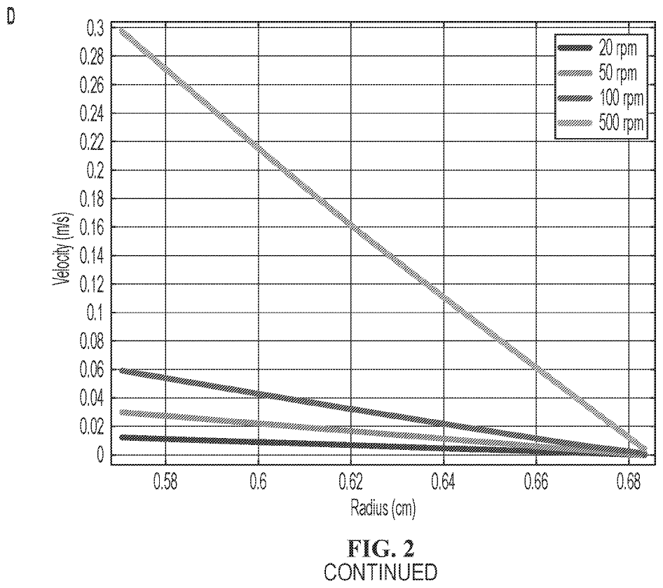

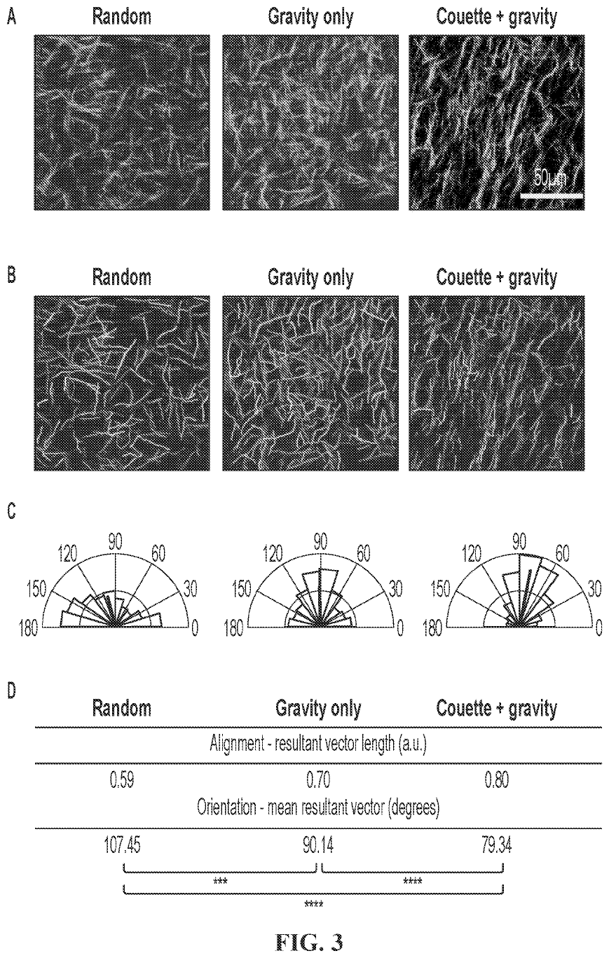

To determine an optimum rotation speed for collagen nucleation and validate our device design to create a laminar Couette flow, we performed a computational fluid dynamics simulation on COMSOL Multiphysics. The simulation results with a rotating inner cylinder showed laminar Couette flow without turbulent Taylor vortices at 50 rpm rotation and up to 500 rpm, the highest rotation speed limit of the motor (FIGS. 2A and 2B). Our finding was consistent with previous studies showing a stable laminar flow without turbulence at a low rotation speed [48]. On glass substrates, an intermediate shear rate between 20 to 80 s⁻¹ has been demonstrated to best align collagen fibers (3 mg/ml) compared to a lower shear rate of 9 s⁻¹ or a higher shear rate of 500 s⁻¹ [32]. Based on our simulation results of shear rate at different rotational speeds, we determined the inner cylinder rotation speed at 50 rpm to achieve the desired shear rate of 35 s⁻¹ (FIG. 2C) and flow velocity of 0.03 m/s (FIG. 2D) on the surface of the inner cylinder. We measured the desired rotation speed using shear rate, rather than shear force, for the following reasons: shear rate does not change as concentration or viscosity changes, and shear rate independently increases collagen nucleation by excluding flow rate as a confounding factor [32].

Couette flow was stopped before any gross gel solidification could be observed. After the 2-minute rotation period, the collagen began its elongation phase under the influence of vertical gravitational force. During this elongation phase, collagen fibers grew along vertical gravitational force after stopping the 2-minute rotation of the inner cylinder. No bulk movement of the gel was seen during the rotation- or gravity-driven phase. An intact tube-shaped gel was formed after a 2-minute rotation and an additional 20-minute gelling period in a stationary condition (FIG. 1E). In contrast, a 10-minute or a 5-minute rotation did not form an intact gel. Instead, fragmented collagen debris was formed even after a 60-minute gelling in a stationary condition (Supplementary FIG. 1A). After generating an intact tube-shaped gel, we cut and spread the gel to make a large rectangular collagen matrix gel (35 mm×25 mm×0.5 mm) (FIG. 1E). Gravity only and a random condition were used as control groups. In the gravity only group, collagen was polymerized in the coaxial cylinder system in a stationary condition without the initial 2-minute rotation. In the random group, collagen pregel solution was poured onto a glass slide with a polydimethylsiloxane (PDMS) frame designed to have the same dimension as the space between two cylinders in the coaxial cylinder system (FIG. 9B). The collagen matrices were gelled on glass slides in a stationary condition to form randomly oriented collagen fibers.

### 3. Collagen Fiber Alignment is Enhanced by Laminar Couette Flow Followed by Gravitational Force

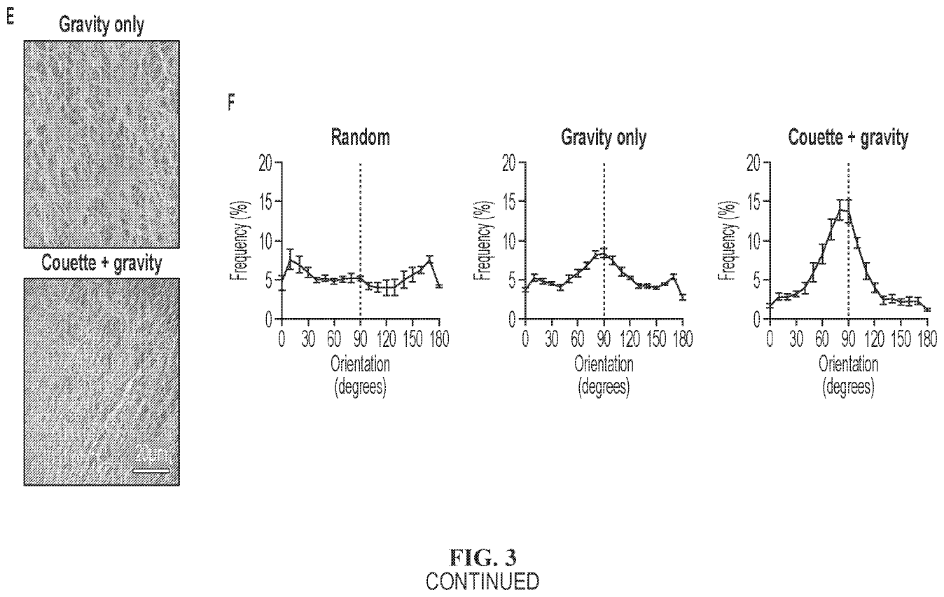

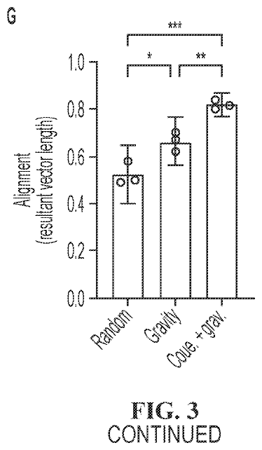

Collagen fibers in 3D matrix gels formed under different experimental conditions were visualized by a multiphoton SHG microscope (FIG. 3A) and computationally segmented using CT-FIRE, a MATLAB-based program (FIG. 3B). We compared the alignment and orientation of collagen fibers in the 3D matrix gel formed in different experimental conditions. First, we analyzed the directional statistics of collagen fibers in 20 randomly picked spots from one gel for each experimental condition. The angular frequency distribution analysis of collagen fibers demonstrated that resultant vector length was higher in the Couette+gravity group (0.8) than in the gravity only (0.7) or random (0.59) groups. (FIGS. 3C and 3D). The fiber orientation angles were also significantly different between the 2-minute rotation group (79.34°) and the no rotation group (90.14°) (FIGS. 3C and 3D). These results suggested that the additional laminar Couette flow promoted fiber alignment and contributed to the fiber orientation deviation (p<0.0001). In addition, SEM images confirmed more aligned but oblique collagen fibers in the Couette+gravity group compared to the gravity only group (FIG. 3E). Our findings, along with previous studies, suggest the orientation of collagen fibers was the combined consequence of the flow direction in both nucleation and elongation phases [32].

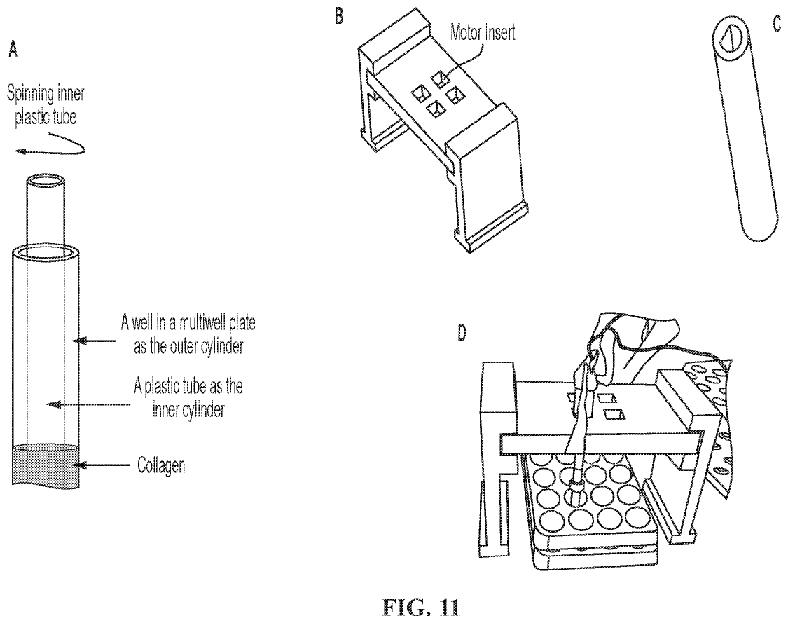

To validate the reproducibility of our method, we performed three independent experiments and investigated the fiber alignment by determining the angular frequency distribution (FIG. 3F) and resultant vector length (FIG. 3G) of collagen fibers. The peak frequency distribution was higher in the Couette+gravity group (13.91% at 80°) than in the gravity only (8.33% at 90°) and random (7.59% at 170°) groups (FIG. 3F). Resultant vector length analyses demonstrated that applying both Couette flow and gravity significantly enhanced fiber alignment in comparison to gravity only (p<0.01) and random (p<0.001) groups (FIG. 3G). As a second measure of fiber alignment, alignment indexes derived from the frequency distribution were also significantly higher in the Couette+gravity group than in gravity only and random groups (FIG. 8C). The alignment and orientation analysis results demonstrated the reproducibility of our novel technology to generate a large collagen matrix gel with homogeneously aligned fibers.

### 4. Each Tumor Spheroid is Surrounded by Radially Aligned and Circumferentially Oriented or Aligned Collagen Fibers in Dual Topographical Tumor Model

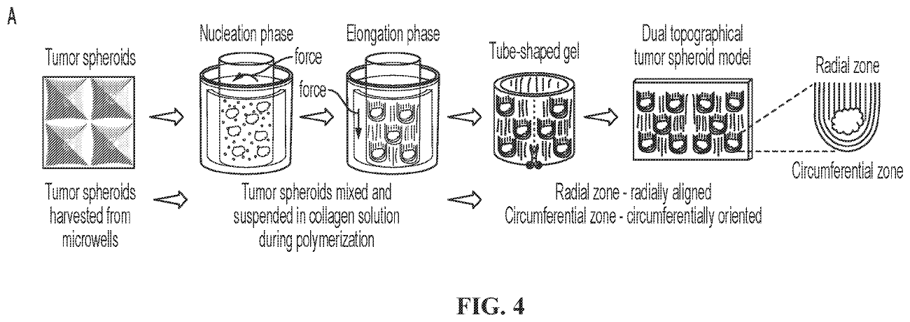

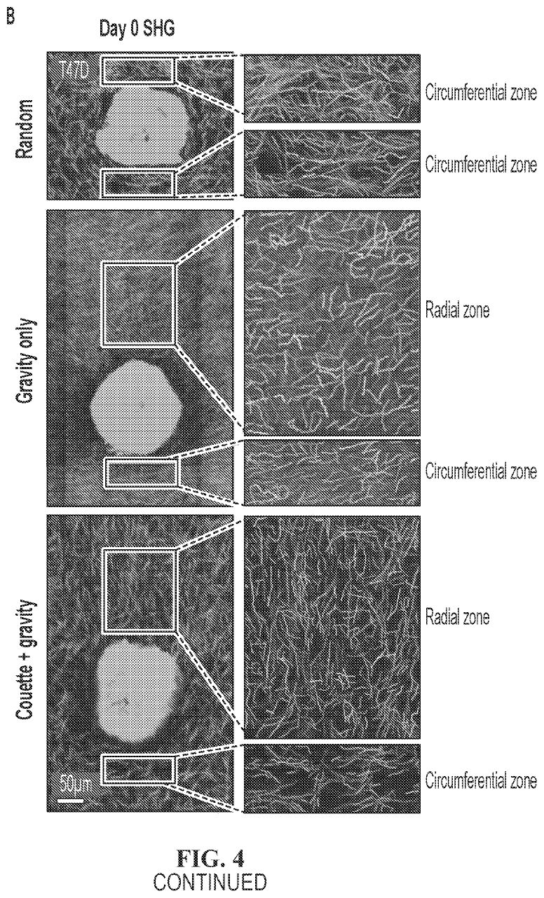

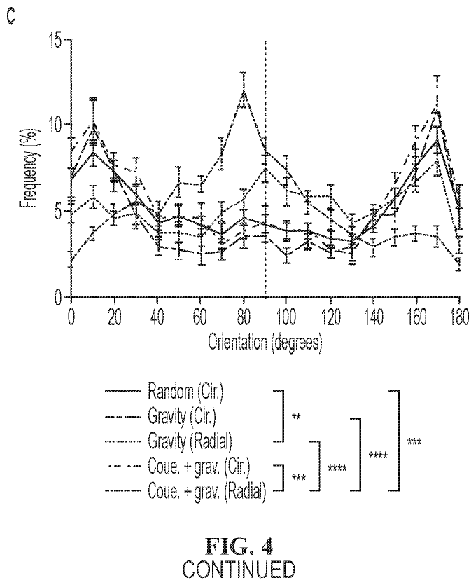

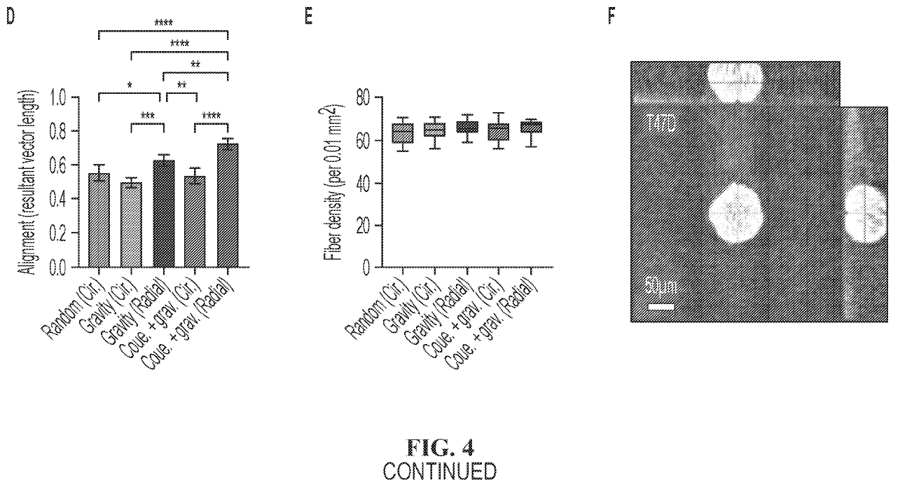

We pre-seeded tumor spheroids within the collagen solution before gelling to allow the close contact of tumor spheroids with the in vivo tumor-like ECM topography. Tumor spheroids of MCF7 and T47D breast cancer cells were uniformly generated in a spherical shape in microwells (FIG. 4A and FIG. 9A). Approximately 1500 tumor spheroids were suspended in 1 mL of collagen solution to achieve a density of two tumor spheroids per mm³. Then, the mixture of collagen solution and tumor spheroids in the coaxial cylinder system underwent 2 minutes of laminar Couette flow and 20 minutes of gravitational force to align collagen fibers (FIG. 4A and FIG. 9B). Immediately after collagen gels were formed, we analyzed the collagen fiber orientation surrounding tumor spheroids using SHG microscopy. Interestingly, collagen fibers surrounding tumor spheroids exhibited a location-specific topography, in which collagen fibers above spheroids were radially aligned, and fibers beneath spheroids were circumferentially oriented or aligned (FIGS. 4B bottom and 4C). This location-specific dual topography was not observed in the collagen gel without tumor spheroids or in random groups with tumor spheroids (FIGS. 4B top and 4C), where collagen was gelled with spheroids on glass slides. In comparison, collagen fibers polymerized only by gravitational force without Couette flow exhibited weaker radial alignment above spheroids (FIGS. 4B middle, 4C, and 4D). Therefore, we concluded that Couette flow with subsequent gravitational force significantly enhanced fiber alignment in the radial zone above tumor spheroids.

To prove that this location-specific topography is not due to tumor cell contractility, we embedded 200 μm glass microbeads instead of tumor spheroids. We found the same location-specific topography where fibers above microbeads were radially aligned, and fibers beneath microbeads were circumferentially oriented or aligned. This result ruled out the possibility that the location-specific topography was due to tumor cell contractility (FIGS. 9D and 9E). Furthermore, there was no difference in fiber density between the radial zone above spheroids and the circumferential zone beneath spheroids (FIG. 4E), which suggests that the circumferentially oriented or aligned topography beneath tumor spheroids was more likely due to spheroids' interference with collagen fiber elongation, rather than aggregated fibers by spheroid weight. The interference of fiber orientation by objects during fiber elongation was reported in a previous study in which cylindrical microposts in a microfluidic device interfered with the fluid flow-guided fiber alignment [21]. No difference in fiber density, suggesting similar mechanical properties [49] between radial and circumferential zones, also suggests that matrix topography can serve as an independent factor in determining tumor invasion patterns. Finally, we verified that the 2-minute Couette flow did not affect the morphology and border complexity of tumor spheroids, indicating that the shear forces from Couette flow did not disrupt tumor spheroid integrity (FIG. 4F and FIG. 9C). Our dual topographical tumor model enables spheroids to be in contact with two most common tumor ECM topographical structures, which recapitulates in vivo tumor microenvironment and makes this model applicable to investigate collective cancer invasion.

### 5. Radially Aligned ECM Topography Promotes a Cluster-Based Cancer Invasion

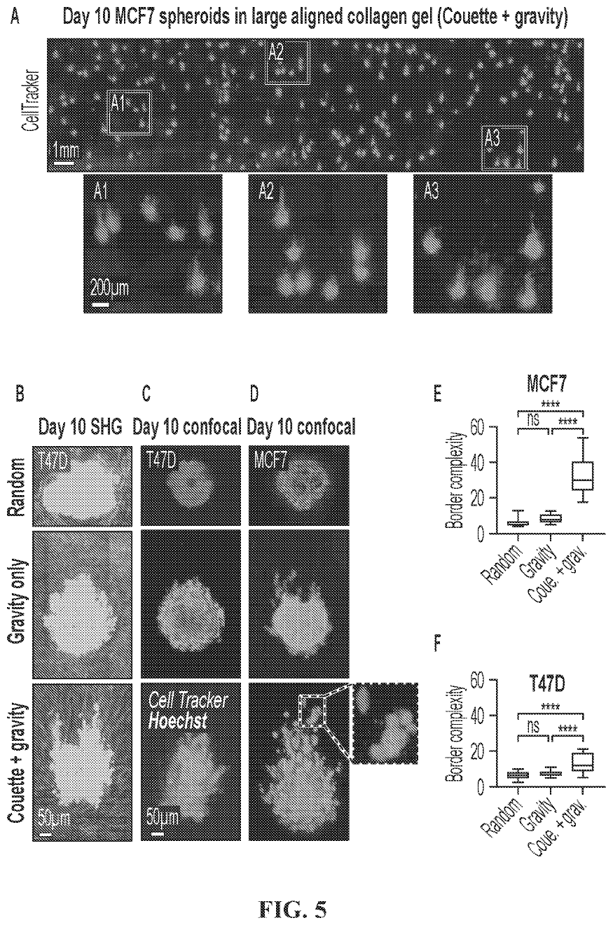

Tumor invasion patterns were analyzed after a 10-day interaction of T47D and MCF7 tumor spheroids with the predisposed collagen fiber structure. Images of MCF7 tumor spheroids after a 10-day invasion in dual topographical model demonstrated evenly distributed tumor spheroids and the same invasion pattern toward radially aligned fibers (FIG. 5A). SHG images of T47D cells demonstrated that the ECM topography on day 10 (FIG. 5B) remained similar to the predisposed topography on day 0 (FIG. 4B). Fibers in the radial zones retained radially aligned structure, and fibers at the circumferential zones were still circumferentially oriented or aligned. No prominent remodeling of predisposed collagen fiber structure was observed. Both SHG and confocal images revealed multicellular disseminated clusters and finger-like projections invading along radially aligned collagen fibers but not in circumferentially oriented or aligned fibers (FIGS. 5C and 5D). In the fibers polymerized only by gravitational force without Couette flow, fewer multicellular disseminated clusters and finger-like projections were observed along the weakly aligned fibers (FIGS. 5C and 5D). Border complexity analysis confirmed that spheroids in Couette+gravity groups had a more irregular border compared to the gravity only and random groups (p<0.0001 for both MCF7 and T47D) (FIGS. 5E and 5F). In random groups where breast cancer spheroids were surrounded entirely by circumferentially oriented or aligned collagen fibers, tumors formed ductules recapitulating well-differentiated human breast cancer. In contrast, the disseminated multicellular clusters and projections observed at the tumor border along radially aligned fibers recapitulated the morphology of poorly differentiated breast cancer.

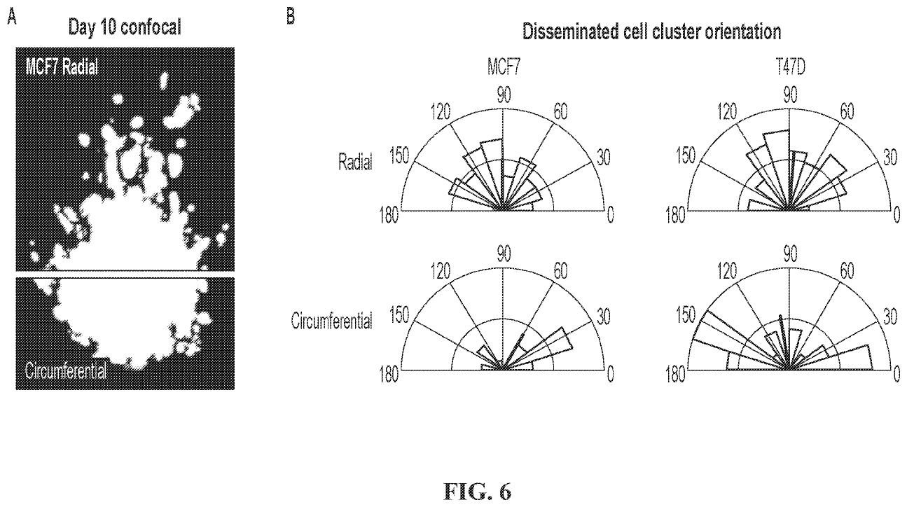

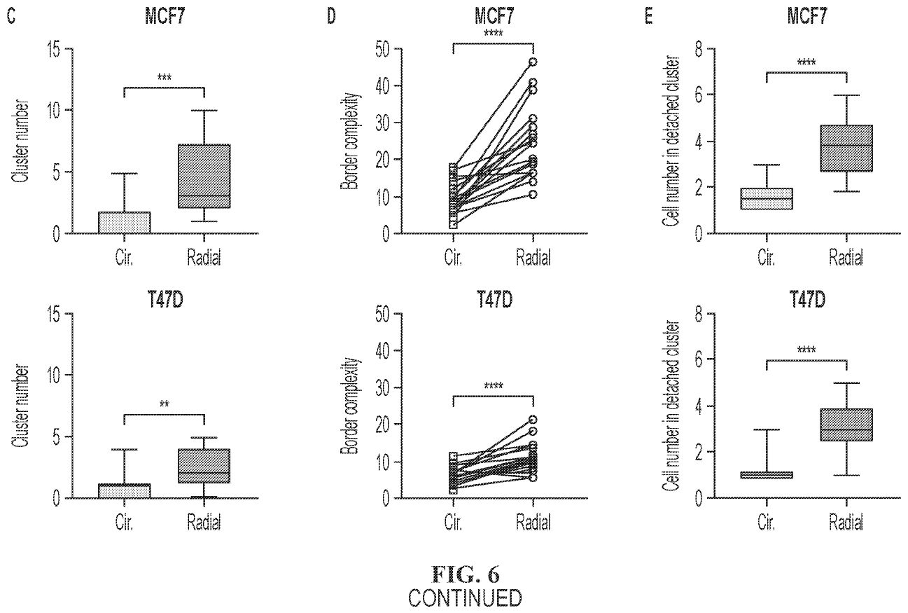

We examined the intra-spheroid morphology responding to two distinct ECM structures, circumferentially orientated or radially aligned (FIG. 6A). The orientation analysis indicated that multicellular clusters disseminated from tumor spheroids invaded and elongated along the collagen fiber direction (FIG. 6B). The side of tumor spheroids interacting with radially aligned fibers had more disseminated cell clusters (p<0.001 for MCF7 and p<0.01 for T47D) (FIG. 6C) and a more irregular border compared to the other side of tumor spheroids interacting with circumferentially oriented or aligned fibers (p<0.0001 for both MCF7 and T47D) (FIG. 6D). The average cell number in disseminated cell clusters was higher in radially aligned fibers (MCF7: 3.76, T47D: 3.08) than in circumferentially oriented or aligned fibers (MCF7: 1.43, T47D: 1.35) (FIG. 6E).

Our aligned collagen gel can also be applied to investigate individual cell behaviors. When cancer cells were seeded as individual cells in collagen gels aligned by Couette flow and gravity, individual cells were surrounded by aligned fibers instead of dual topography. After a 7-day culture in aligned fibers, tumor cells formed elongated multicellular clusters along the orientation of aligned fibers, similar to the disseminated cell clusters in dual topographical spheroid models (FIG. 10). In summary, our dual topographical tumor spheroid model with disseminated tumor clusters invades along radially aligned fibers and recapitulates the histology of tumor invasion in human cancer.

### 6. Dual Topographical Tumor Model Distinguishes Invasion Pattern of Tumor Spheroids and Organoids

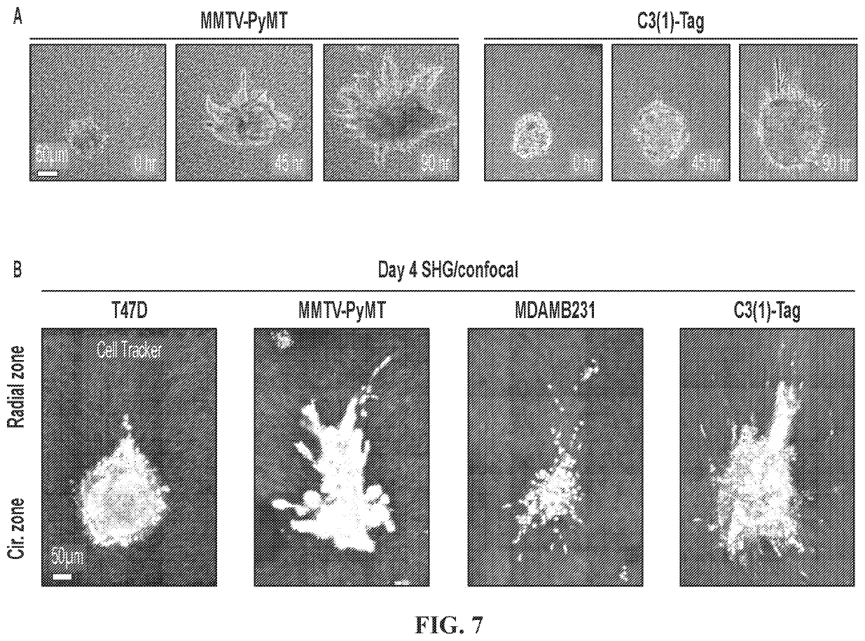

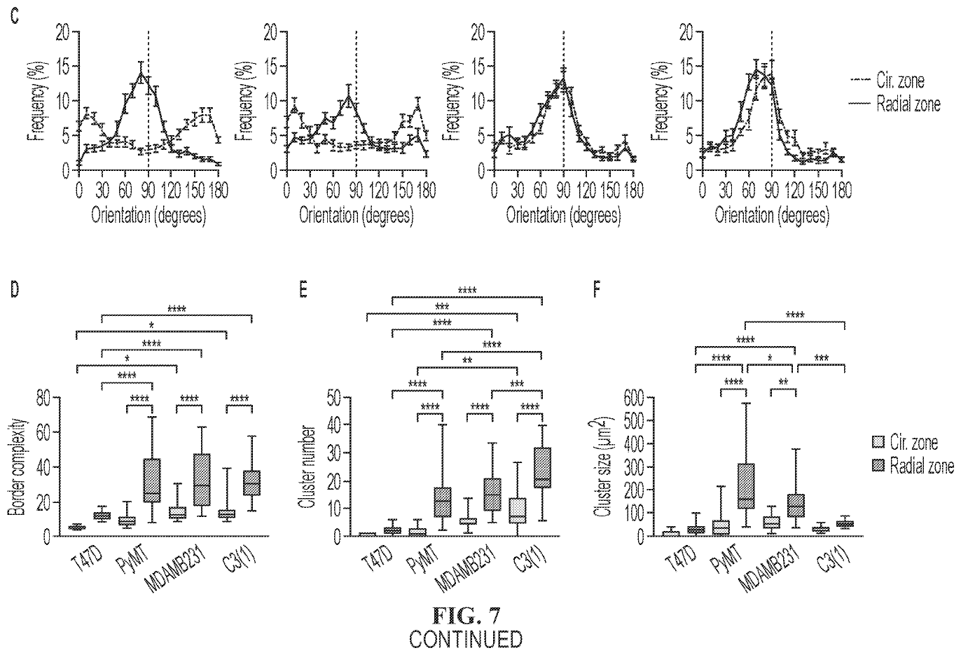

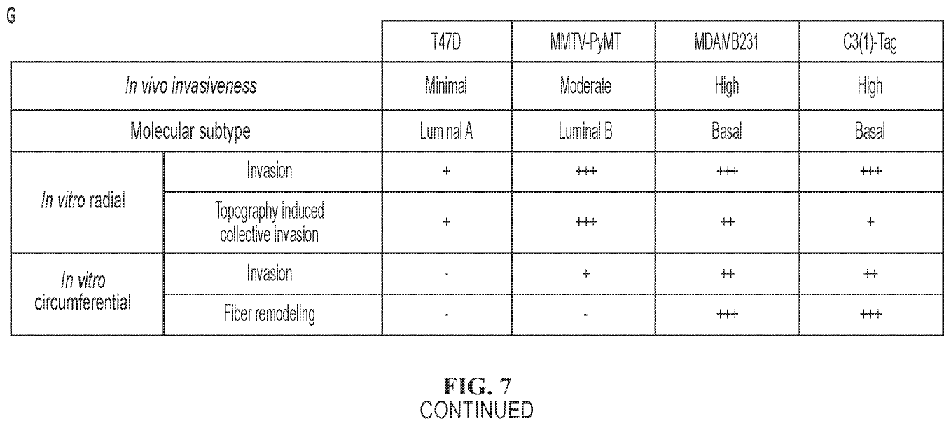

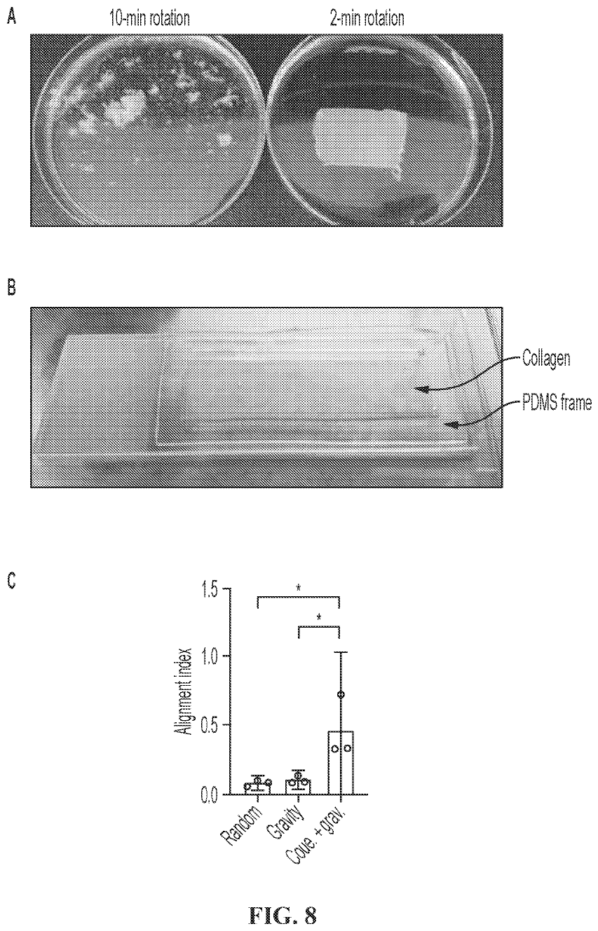

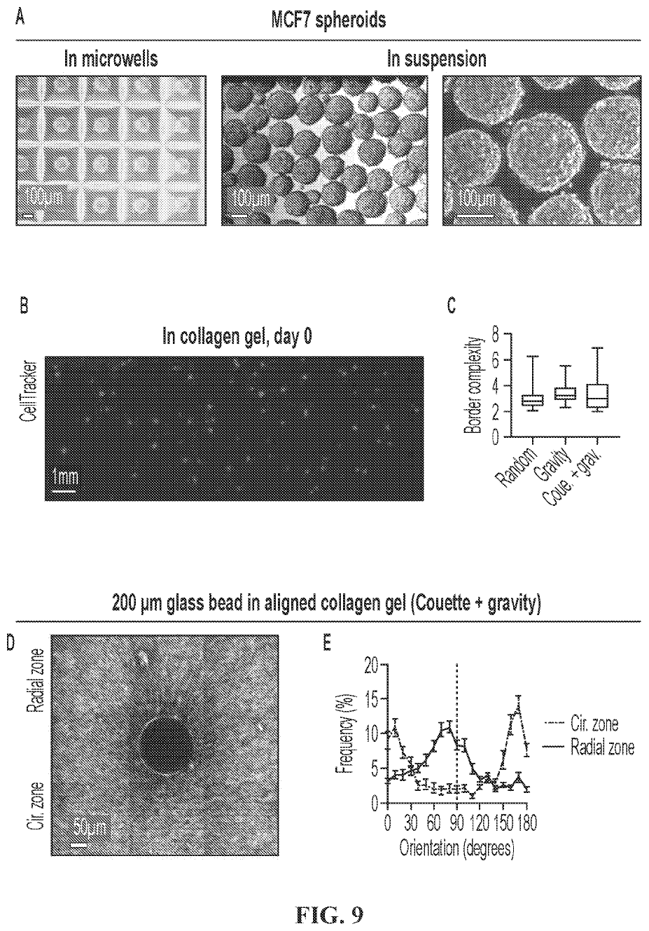

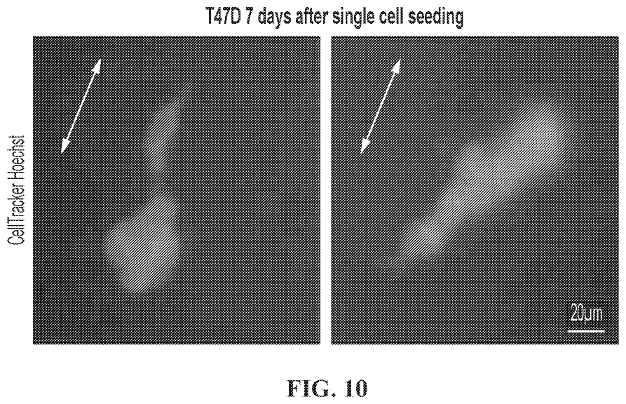

To evaluate whether our dual topographical model can distinguish tumors with different invasive and metastatic potentials, we investigated breast tumor spheroid or organoid models differing in ER/PR/HER2 status and invasion ability. Tumor spheroids were originated from T47D and MDAMB231 human breast cancer cells. Tumor organoids were derived from mouse mammary tumor models, MMTV-PyMT [41] and C3(1)-Tag [42]. T47D represents a luminal A (ER⁺/PR^{+/-}/HER2⁻) subtype and is minimally invasive [50, 51]. MMTV-PyMT represents a luminal B (ER⁺/PR^{+/-}/HER2⁺) subtype and is moderately invasive. Both MDAMB231 and C3(1)-Tag represent the basal triple-negative (ER⁻/PR⁻/HER2⁻) subtype are highly invasive in vivo [52]. After four days of invasion in our dual topographical tumor model, tumor spheroids/organoids from the different models displayed different invasion patterns responding to local fiber structures. In radial zones, all four tumor spheroid/organoid models invaded with finger-like projections and disseminated cell clusters along radially aligned fibers (FIGS. 7A and 7B). Fiber orientation in radial zones retained radial alignment in all four tumor spheroids/organoids (FIG. 7C). Moreover, MMTV-PyMT, MDAMB231, and C3(1)-Tag spheroids/organoids, which are metastatic in vivo, had significantly higher border complexities and more disseminated cell clusters compared to T47D spheroids, which is non-metastatic in vivo (FIGS. 7D and 7E). In the circumferential zone, basal-like tumor spheroids/organoids, MDAMB231 and C3(1)-Tag remodeled collagen fibers to be radially aligned (FIG. 7C). On the contrary, T47D and MMTV-PyMT spheroids/organoids failed to remodel circumferentially oriented or aligned structures. In the circumferential zone, MDAMB231 and C3(1)-Tag spheroids/organoids demonstrated higher border complexity than T47D spheroids (FIG. 7D). C3(1)-Tag organoids had more disseminated cell clusters than T47D and MMTV-PyMT spheroids/organoids (FIG. 7E).

Allowing tumor spheroids/organoids to interact with both radially aligned and circumferentially oriented topography, our dual topographical tumor model further revealed how different tumors react uniquely to local topography. Compared to the circumferentially oriented or aligned side, radially aligned topography significantly increased border complexity and disseminated cell cluster number in MMTV-PyMT, MDAMB231, and C3(1)-Tag spheroids/organoids and T47D spheroids (FIGS. 7D and 7E). Interestingly, while this enhancement induced by radially aligned topography was observed in all the four tumor spheroids/organoid models, topography-induced collective invasion was more significant in MMTV-PyMT and MDAMB231 spheroids/organoids (FIG. 7F). We summarize the in vivo invasiveness, molecular subtypes, and in vitro invasion pattern of the tumor spheroids/organoids tested in our dual topographical tumor model in FIG. 7G.

### Discussion

Extracellular matrix (ECM), the natural scaffold surrounding tumors, influences cancer cell behavior. A readily fabricated model recapitulating the interaction between tumors and ECM structures is of great interest in understanding how ECM regulates tumor invasion and identifying invasion-specific therapeutic targets. In the present study, we develop a topographical matrix by applying distinct forces specific for each collagen polymerization phase to align collagen fibers. Our 3D dual topographical tumor model enables each tumor spheroid to be surrounded by radially aligned and circumferentially oriented or aligned fibers, the two most common topographical features of tumor stroma.

Aligning matrix fibers has gained much interest in the last few decades for its broad application in recapitulating the ECM topography. Properly aligned fibers represent the physiological ECM scaffold features such as heart and skeletal muscle and the pathological features in the tumor microenvironment. However, previous methods of aligning fibers have limitations in their application as 3D tumor spheroid models (see Table 1 below). The cellular contraction method, which aligns collagen fibers by fibroblast-induced strain, requires the decellularization of fibroblasts before seeding target cells [18, 19]. The decellularization step also makes the fabrication process time-consuming and induces potential cytotoxicity in the gel. Electrospinning has been widely used to generate aligned fibers made of natural and artificial materials [53] but requires a bulky machine and cytotoxic crosslinkers [54, 55]. Also, the pore size of densely compacted electrospun fiber scaffolds is too small to embed tumor spheroids [56]. Tumor spheroids can only be seeded onto the fiber sheet surface with a limited number of cells contacting the matrix topography. Techniques used to increase the pore size between electrospun fibers such as salt leaching [57] and sacrificial fiber [58] may change the material properties. Magnetic beads embedded in collagen gels pulled by an external magnetic field to guide fiber assembly direction is another method to align fibers [29]. However, the cytotoxicity and autofluorescence of magnetic beads diminish their application as tumor models [59]. Fluid flow is another commonly applied method to align fibers [31-33]. The shear force generated by laminar flow in the microfluidic devices helps control the anisotropic elongation of fibers [32]. However, the flow in microscale channels may be significantly disturbed by tumor spheroids, limiting the usage of microfluidics as tumor spheroid models. Finally, although the extensional strain method generates a highly aligned collagen sheet, the collagen layer is coated on thin films. Tumor spheroids cannot be embedded to create a 3D model on such a thin collagen layer [24, 60]. Extensional strain driven by a rotating acupuncture needle in a polymerized collagen gel generates radially aligned fibers centering on the needle [61, 62]. However, highly aligned fibers are only seen in the area close to the needle. The fiber directionality decreases with distance from the needle, making homogeneous alignment difficult [62]. Our 3D dual topography system has several advantages to overcome the limitations of these conventional methods. First, the phase-specific forces we apply to enhance the fiber alignment are achieved within the same device without time delay or sample transfer between devices. Second, our method does not require additional reagents or post-polymerization treatment, thus a cytotoxic-free large-scale collagen gel with anisotropic aligned fibrils can be rapidly generated. Third, a coaxial rotating cylinder system allows pre-seeded tumor spheroids to be surrounded directly by predisposed structures without damaging fiber architecture. Furthermore, our method to fabricate aligned collagen gel can also be applied beyond cancer research. For example, our approach has potential in large-scale tissue engineering which aligned structure is required or in recapitulating tube-shaped organs such as the cardiovascular system.

Studies based on 3D hydrogel models [18, 34] or quasi 3D topographical substrates [63, 64] reported that radially aligned matrix topography enhanced tumor invasion and migration. In these models, however, tumor spheroids were not closely surrounded by the predisposed matrix structures, a unique histological pattern of tumor invasion in human cancer. For example, tumor invasion was not observed in weakly invasive breast tumors such as MCF7 and T47D cells in a hydrogel model, which has pre-aligned collagen fibers only in a restricted area [20]. In a recent study [65], MCF7 spheroids invaded in laser-ablated microtracks in dense collagen, the interface between collagen and culture dish, fibroblast-rich dense collagen, and randomly oriented low-density collagen (1.6 mg/ml). Guidance cues are presented to study cancer invasion in response to ECM microarchitecture in both previous [65] and our present study. The previous study focuses on generating confined space to show that high ECM confinement rescues cell-cell junctions and leads to collective invasion [65]. By comparison, our 3D model is characterized by two different predisposed ECM structures, radially aligned and circumferentially oriented or aligned fibers and it demonstrates that tumor matrix topography is a determining factor in cancer invasion of both highly invasive and weakly invasive breast tumors. Moreover, cancer cells are known to react with local mechanical properties such as stiffness [66]. Our model places each tumor spheroid/organoid in direct contact with two different ECM structures at the same local fiber density within the same gel to provide direct proof of topography-induced collective cancer invasion.

Recent studies revealed that tumors invade as multicellular clusters by retaining E-cadherin expression to carry more metastatic potential [67-69]. In histopathology of human cancer, tumor cluster dissemination or tumor budding is defined by cell clusters of usually less than four or five tumor cells breaking apart from the main tumor [70]. Tumor budding is correlated with poor prognosis, larger tumor size, frequent lymph node metastasis, and distant metastasis in breast cancer [71], colorectal cancer [70], pancreatic cancer [72], gastric cancer [73], and other cancer types [74, 75]. More importantly, a recent prospective randomized controlled study reported that cancer patients with tumor budding have significantly higher tumor recurrence rates when treated with surgery alone compared to additional postoperative chemotherapy [76]. These findings indicate the clinical implication of disseminated tumor clusters in deciding treatment strategies for cancer patients.

Tumor cluster dissemination in aligned breast tumor stroma is a metastasis precursor, however, mechanisms for the formation of disseminated tumor clusters are unclear. Partial epithelial-mesenchymal transition (EMT) is a generally accepted mechanism of tumor budding [77]. Instead of undergoing a complete EMT, cancer cells have the plasticity of retaining both epithelial and mesenchymal characteristics to invade as small cell clusters [77]. Our current study demonstrates that radially aligned topography acts as an external biophysical cue that promotes tumor cluster dissemination in luminal A (T47D), luminal B (MMTV-PyMT), and basal (MDAMB231 and C3(1)-Tag) subtypes of breast cancer. In mesenchymal-type tumor cells, a high-density ECM caused cell jamming and facilitated cluster formation [78]. However, in our dual topographical model, both radially aligned and circumferentially oriented or aligned fibers are within the same gel and have the same fiber density. Therefore, ECM topography can be an independent factor in driving collective cell invasion and cell cluster dissemination in both epithelial and mesenchymal-type cancer and may have distinct mechanisms other than EMT or cell jamming. Several possible mechanisms include Rho/ROCK signaling of cell contractility, cell-cell adhesion regulation aside from EMT, or integrin mechanotransduction involved in cell-matrix interaction. Rho/ROCK signaling-mediated cell contractility was shown to play an essential role for tumors to align surrounding matrix fibers [17, 79]. But after the fibers are remodeled, the invasion of MDAMB231 cells in aligned fibers no longer needs Rho/ROCK mediated contractility [17]. It is not clear whether the invasion of weakly invasive tumors such as T47D and MCF7 in pre-aligned fibers is independent of Rho/ROCK signaling. An alternative mechanism of collective cluster dissemination is an activation of the developmental pathway. A re-acquired expression of adhesion molecules by plakoglobin [68], keratin 14 [69], or CD44 upregulation [80] holds tumor cells together and increases the survival of tumor clusters in circulation. Cancer cell invasion in ECM fibers is also extensively affected by the integrin-regulated interaction between cells and collagen fibers [81]. It is still unknown whether and how cell-cell adhesion or cell-matrix adhesion mediates topography-induced collective invasion. These unresolved questions reinforce our new 3D model as a in vivo-like platform for elucidating the mechanism of topography-induced tumor cluster dissemination.

We developed a rapid and reproducible method to fabricate a 3D dual topographical tumor model in which tumor spheroids are surrounded by two common predisposed tumor ECM structures, radially aligned and circumferentially oriented or aligned collagen fibers. Radially aligned topography promotes multicellular cluster-dissemination detached from the main tumor, which recapitulates in vivo cancer invasion. Our 3D tumor model is a viable experimental platform for investigating tumor invasion and identifying therapeutic targets against metastasis.

### Example 2: Design and Assembly of a Coaxial Rotating Cylinder System with Multiwell Plate

Our coaxial rotating cylinder system comprises of a 3D printed motor holder, a motor, a 3D printed connector for the motor shaft, a plastic tube as the inner cylinder, and a multiwell plate. Each well of a multiwell plate serves as the outer cylinder. The motor holder and the connector concentrically align the inner and outer cylinders. The rotation of the inner glass cylinder was powered by a direct current 6 volt 500 revolutions per minute (rpm) micro speed reduction motor, and the rotation speed was controlled by a pulse-width modulation stepless direct current motor speed controller. The plastic was connected to the motor shaft by a customized 3D-printed connector. The 3D printed parts were designed using Autodesk Inventor software (Autodesk, USA) and printed by a desktop 3D printer with acrylonitrile butadiene styrene filaments. After being concentrically aligned, the smaller inner cylinder was placed inside the center of the larger outer cylinder leaving an empty annulus between the two cylinders for collagen gelling.

Fabrication of Collagen Matrices with Aligned Fibers

Collagen matrices were prepared by mixing type I rat tail telocollagen solution and neutralization solution in a ratio of 9:1 (RatCol® Rat Tail Collagen for 3D Hydrogels, Advanced BioMatrix, USA). The volume of collagen added between the empty annulus between the two cylinders is adjustable according to each well's volume in a multiwell plate. A 2-minute rotation of the inner cylinder at 50 rpm was applied to generate Couette flow for collagen monomer nucleation on the glass surface and followed by a 20-minute gelling in a stationary condition for the gravitational force to guide collagen fiber elongation. Collagen was polymerized at room temperature.

### Example 3: Preferred Multiwell Format with Multiple Coaxial Cylinders

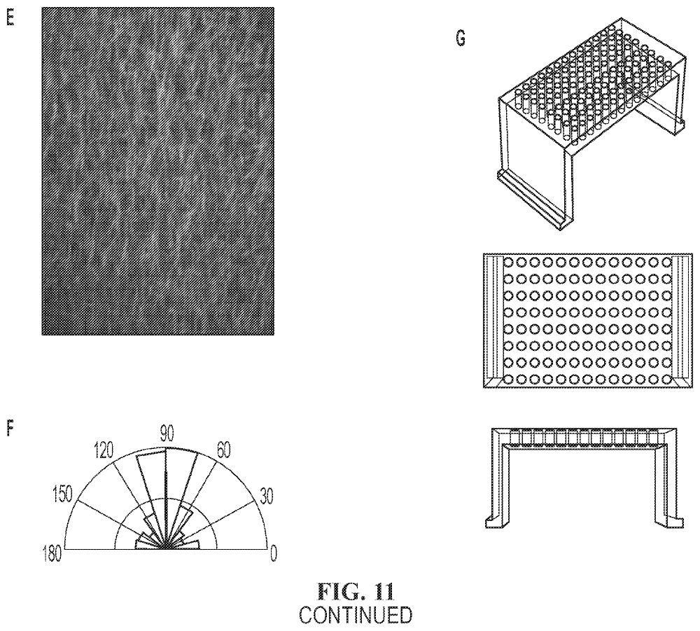

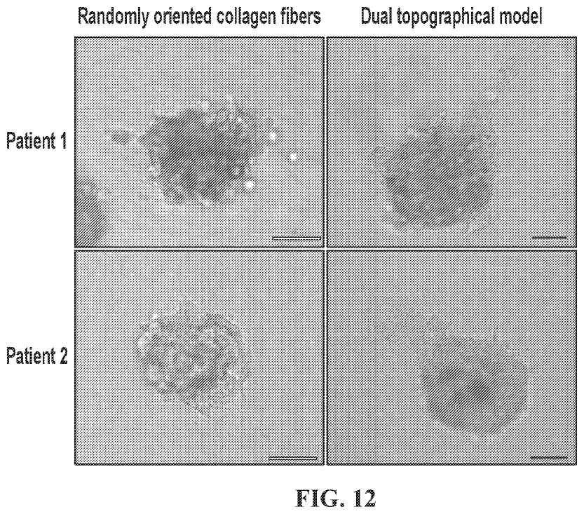

FIG. 11 shows a preferred multiwell format system. In FIG. 11A: a set of coaxial cylinders in a multiwell system. In a multiwell system, each well in a multiwell plate serves as the outer cylinder and a plastic tube as the inner cylinder. The spinning of the inner plastic tube aligns the collagen fibers by a phase-specific, force-guided method. FIGS. 11B-11C: A computer-aided design of (B) a motor holder and (C) a connector for the motor shaft of a coaxial cylinder system. FIG. 11D depicts a representative image of a multiwell coaxial cylinder system consists of a 3D printed motor holder, a motor, a connector for the motor shaft, a plastic tube as the inner cylinder, and a 24 well plate. The inner and outer cylinders are concentrically aligned by the motor holder and the connector. FIG. 11E: A multiphoton second-harmonic generation image shows aligned collagen fibers generated by a 24-well multiwell coaxial cylinder system. FIG. 11F: The angular frequency distribution of collagen fibers demonstrates highly aligned collagen fibers with a peak orientation around 90°. Red line represents the magnitude and direction of mean resultant vectors. FIG. 11G: A computer-aided design of motor holder designed for a 96-well coaxial cylinder system.

### Example 4

The disclosed 3D biomimetic tumor model to patient-derived pancreatic cancer organoid. In the 3D biomimetic tumor model, each patient-derived pancreatic cancer organoid surrounded by radially aligned fibers on one side and circumferentially oriented or aligned fibers the opposing side. Different invasion patterns between the side of radially and the opposing side of circumferentially oriented or aligned fibers, indicating the 3D biomimetic tumor model can be used to analyze invasion patterns for patient-derived tumor organoids.

### Example 5: Drug Screening

To test the effects of ECM topography on cancer cell response to oncology drugs, we will use the dual topographical tumor organoid multiwell system for drug screening. First, tumor organoids will be mixed and seeded together within a collagen pregel solution at a 1:9:1 ratio (one part of tumor organoids in culture media, nine parts of type I collagen solution, and one part of neutralization buffer) (RatCol® Rat Tail Collagen for 3D Hydrogels, Advanced BioMatrix, USA), and poured into the space between the two coaxial cylinders. Then a 2-minute laminar Couette flow driven by inner cylinder rotation followed by a 20-minute gravity-driven fiber elongation will polymerize collagen at room temperature. Next, the resulting tube-shaped collagen gels embedded with tumor organoids will be cut and spread out to form dual topographical tumor models. Finally, the gel in each well of a multiwell system will be transferred to a regular multiwell plate. Each drug in a drug library will be distributed and diluted into appropriate wells by an automated simultaneous pipettor (CyBi-well 96-Channel Simultaneous Pipettor, CyBio, Germany) to yield the final concentration of 1 μM in the culture medium. After 72-hour drug treatment, cell viability was assessed using PrestoBlue HS Cell Viability Reagent (Thermo Fisher Scientific, USA) on a microplate reader (CLARIOstar Plus, BMG Labtech, Germany). All drugs will be ranked by their Z-score of cell viability to select the drug hits that most inhibit topography-induced cancer cell dissemination.

### Example 6

Pancreatic ductal adenocarcinoma organoids from two different patients were produced. Results are shown in *

### REFERENCES

[1] F. Kai, A. P. Drain, V. M. Weaver, The Extracellular Matrix Modulates the Metastatic Journey, Dev Cell 49(3) (2019) 332-346.
[2] M. Giussani, G. Merlino, V. Cappelletti, E. Tagliabue, M. G. Daidone, Tumor-extracellular matrix interactions: Identification of tools associated with breast cancer progression, Semin Cancer Biol 35 (2015) 3-10.
[3] S. Kaushik, M. W. Pickup, V. M. Weaver, From transformation to metastasis: deconstructing the extracellular matrix in breast cancer, Cancer Metastasis Rev 35(4) (2016) 655-667.
[4] A. R. Chin, S. E. Wang, Cancer Tills the Premetastatic Field: Mechanistic Basis and Clinical Implications, Clin Cancer Res 22(15) (2016) 3725-33.
[5] Y Liu, X. Cao, Characteristics and Significance of the Pre-metastatic Niche, Cancer Cell 30(5) (2016) 668-681.
[6] P. Friedl, S. Alexander, Cancer invasion and the microenvironment: plasticity and reciprocity, Cell 147(5) (2011) 992-1009.
[7] T. R. Cox, J. T. Erler, Remodeling and homeostasis of the extracellular matrix: implications for fibrotic diseases and cancer, Dis Model Mech 4(2) (2011) 165-78.
[8] R. Malik, P. I. Lelkes, E. Cukierman, Biomechanical and biochemical remodeling of stromal extracellular matrix in cancer, Trends Biotechnol 33(4) (2015) 230-6.
[9] K. M. Riching, B. L. Cox, M. R. Salick, C. Pehlke, A. S. Riching, S. M. Ponik, B. R. Bass, W. C. Crone, Y Jiang, A. M. Weaver, K. W. Eliceiri, P. J. Keely, 3D collagen alignment limits protrusions to enhance breast cancer cell persistence, Biophys J 107(11) (2014) 2546-58.
[10] S. Alexander, B. Weigelin, F. Winkler, P. Friedl, Preclinical intravital microscopy of the tumour-stroma interface: invasion, metastasis, and therapy response, Curr Opin Cell Biol 25(5) (2013) 659-71.
[11] A. E. Mayorca-Guiliani, C. D. Madsen, T. R. Cox, E. R. Horton, F. A. Venning, J. T. Erler, ISDoT: in situ decellularization of tissues for high-resolution imaging and proteomic analysis of native extracellular matrix, Nat Med 23(7) (2017) 890-898.
[12] X. J. Ma, S. Dahiya, E. Richardson, M. Erlander, D. C. Sgroi, Gene expression profiling of the tumor microenvironment during breast cancer progression, Breast Cancer Res 11(1) (2009) R7.
[13] M. W. Conklin, R. E. Gangnon, B. L. Sprague, L. Van Gemert, J. M. Hampton, K. W. Eliceiri, J. S. Bredfeldt, Y. Liu, N. Surachaicharn, P. A. Newcomb, A. Friedl, P. J. Keely, A. Trentham-Dietz, Collagen Alignment as a Predictor of Recurrence after Ductal Carcinoma In Situ, Cancer Epidemiol Biomarkers Prev 27(2) (2018) 138-145.
[14] T. R. Lyons, J. O'Brien, V. F. Borges, M. W. Conklin, P. J. Keely, K. W. Eliceiri, A. Marusyk, A. C. Tan, P. Schedin, Postpartum mammary gland involution drives progression of ductal carcinoma in situ through collagen and COX-2, Nat Med 17(9) (2011) 1109-15.
[15] K. R. Levental, H. Yu, L. Kass, J. N. Lakins, M. Egeblad, J. T. Erler, S. F. Fong, K. Csiszar, A. Giaccia, W. Weninger, M. Yamauchi, D. L. Gasser, V. M. Weaver, Matrix crosslinking forces tumor progression by enhancing integrin signaling, Cell 139(5) (2009) 891-906.
[16] H. E. Balcioglu, B. van de Water, E. H. Danen, Tumor-induced remote ECM network orientation steers angiogenesis, Sci Rep 6 (2016) 22580.
[17] P. P. Provenzano, D. R. Inman, K. W. Eliceiri, S. M. Trier, P. J. Keely, Contact guidance mediated three-dimensional cell migration is regulated by Rho/ROCK-dependent matrix reorganization, Biophys J 95(11) (2008) 5374-84.
[18] A. Ray, R. K. Morford, N. Ghaderi, D. J. Odde, P. P. Provenzano, Dynamics of 3D carcinoma cell invasion into aligned collagen, Integr Biol (Camb) 10(2) (2018) 100-112.
[19] A. Ray, Z. M. Slama, R. K. Morford, S. A. Madden, P. P. Provenzano, Enhanced Directional Migration of Cancer Stem Cells in 3D Aligned Collagen Matrices, Biophys J 112(5) (2017) 1023-1036.
[20] W. Han, S. Chen, W. Yuan, Q. Fan, J. Tian, X. Wang, L. Chen, X. Zhang, W. Wei, R. Liu, J. Qu, Y. Jiao, R. H. Austin, L. Liu, Oriented collagen fibers direct tumor cell intravasation, Proc Natl Acad Sci USA 113(40) (2016) 11208-11213.
[21] X. Gong, J. Kulwatno, K. L. Mills, Rapid fabrication of collagen bundles mimicking tumor-associated collagen architectures, Acta Biomater (2020).
[22] Y. Yang, H. Zheng, Y. Zhan, S. Fan, An emerging tumor invasion mechanism about the collective cell migration, Am J Transl Res 11(9) (2019) 5301-5312.
[23] D. Vader, A. Kabla, D. Weitz, L. Mahadevan, Strain-induced alignment in collagen gels, PLoS One 4(6) (2009) e5902.
[24] E. Nam, W. C. Lee, S. Takeuchi, Formation of Highly Aligned Collagen Nanofibers by Continuous Cyclic Stretch of a Collagen Hydrogel Sheet, Macromol Biosci 16(7) (2016) 995-1000.
[25] K. M. Riching, B. L. Cox, M. R. Salick, C. Pehlke, A. S. Riching, S. M. Ponik, B. R. Bass, W. C. Crone, Y Jiang, A. M. Weaver, K. W. Eliceiri, P. J. Keely, 3D Collagen Alignment Limits Protrusions to Enhance Breast Cancer Cell Persistence, Biophysical Journal 107(11) (2014) 2546-2558.
[26] J. A. Matthews, G. E. Wnek, D. G. Simpson, G. L. Bowlin, Electrospinning of collagen nanofibers, Biomacromolecules 3(2) (2002) 232-8.
[27] K. S. Rho, L. Jeong, G. Lee, B. M. Seo, Y. J. Park, S. D. Hong, S. Roh, J. J. Cho, W. H. Park, B. M. Min, Electrospinning of collagen nanofibers: effects on the behavior of normal human keratinocytes and early-stage wound healing, Biomaterials 27(8) (2006) 1452-61.
[28] S. Zhong, W. E. Teo, X. Zhu, R. W. Beuerman, S. Ramakrishna, L. Y. Yung, An aligned nanofibrous collagen scaffold by electrospinning and its effects on in vitro fibroblast culture, J Biomed Mater Res A 79(3) (2006) 456-63.
[29] R. B. Dickinson, S. Guido, R. T. Tranquillo, Biased cell migration of fibroblasts exhibiting contact guidance in oriented collagen gels, Ann Biomed Eng 22(4) (1994) 342-56.
[30] C. Guo, L. J. Kaufman, Flow and magnetic field induced collagen alignment, Biomaterials 28(6) (2007) 1105-14.
[31] B. Lanfer, U. Freudenberg, R. Zimmermann, D. Stamov, V. Korber, C. Werner, Aligned fibrillar collagen matrices obtained by shear flow deposition, Biomaterials 29(28) (2008) 3888-3895.
[32] N. Saeidi, E. A. Sander, J. W. Ruberti, Dynamic shear-influenced collagen self-assembly, Biomaterials 30(34) (2009) 6581-92.
[33] B. Burkel, B. A. Morris, S. M. Ponik, K. M. Riching, K. W. Eliceiri, P. J. Keely, Preparation of 3D Collagen Gels and Microchannels for the Study of 3D Interactions In Vivo, J Vis Exp (111) (2016).
[34] N. Gjorevski, A. S. Piotrowski, V. D. Varner, C. M. Nelson, Dynamic tensile forces drive collective cell migration through three-dimensional extracellular matrices, Sci Rep 5 (2015) 11458.
[35] P. A. Agudelo-Garcia, J. K. De Jesus, S. P. Williams, M. O. Nowicki, E. A. Chiocca, S. Liyanarachchi, P. K. Li, J. J. Lannutti, J. K. Johnson, S. E. Lawler, M. S. Viapiano, Glioma cell migration on three-dimensional nanofiber scaffolds is regulated by substrate topography and abolished by inhibition of STAT3 signaling, Neoplasia 13(9) (2011) 831-40.
[36] M. Gale, M. S. Pollanen, P. Markiewicz, M. C. Goh, Sequential assembly of collagen revealed by atomic force microscopy, Biophys J 68(5) (1995) 2124-8.
[37] C. R. Holloway, R. J. Dyson, D. J. Smith, Linear Taylor-Couette stability of a transversely isotropic fluid, Proceedings of the Royal Society A: Mathematical, Physical and Engineering Sciences 471(2178) (2015) 20150141.
[38] J. S. Bredfeldt, Y. Liu, C. A. Pehlke, M. W. Conklin, J. M. Szulczewski, D. R. Inman, P. J. Keely, R. D. Nowak, T. R. Mackie, K. W. Eliceiri, Computational segmentation of collagen fibers from second-harmonic generation images of breast cancer, J Biomed Opt 19(1) (2014) 16007.
[39] P. Berens, CircStat: A MATLAB Toolbox for Circular Statistics, 2009 31(10) (2009) 21.
[40] G. Razian, Y. Yu, M. Ungrin, Production of large numbers of size-controlled tumor spheroids using microwell plates, J Vis Exp (81) (2013) e50665.
[41] C. T. Guy, R. D. Cardiff, W. J. Muller, Induction of mammary tumors by expression of polyomavirus middle T oncogene: a transgenic mouse model for metastatic disease, Mol Cell Biol 12(3) (1992) 954-61.
[42] I. G. Maroulakou, M. Anver, L. Garrett, J. E. Green, Prostate and mammary adenocarcinoma in transgenic mice carrying a rat C3(1) simian virus 40 large tumor antigen fusion gene, Proc Natl Acad Sci USA 91(23) (1994) 11236-40.
[43] V. Padmanaban, E. M. Grasset, N. M. Neumann, A. K. Fraser, E. Henriet, W. Matsui, P. T. Tran, K. J. Cheung, D. Georgess, A. J. Ewald, Organotypic culture assays for murine and human primary and metastatic-site tumors, Nat Protoc 15(8) (2020) 2413-2442.
[44] Y. Hou, J. Konen, D. J. Brat, A. I. Marcus, L. A. D. Cooper, TASI: A software tool for spatial-temporal quantification of tumor spheroid dynamics, Sci Rep 8(1) (2018) 7248.
[45] T. Oskarsson, Extracellular matrix components in breast cancer progression and metastasis, Breast 22 Suppl 2 (2013) S66-72.
[46] M. K. Jena, J. Janjanam, Role of extracellular matrix in breast cancer development: a brief update, F1000Res 7 (2018) 274.
[47] H. L. Fuchs, A. J. Christman, G. P. Gerbi, E. J. Hunter, F. J. Diez, Directional flow sensing by passively stable larvae, J Exp Biol 218(Pt 17) (2015) 2782-92.
[48] D. Hirshfeld, D. C. Rapaport, Growth of taylor vortices: A molecular dynamics study, Phys Rev E Stat Phys Plasmas Fluids Relat Interdiscip Topics 61(1) (2000) R21-4.
[49] E. Hadjipanayi, V. Mudera, R. A. Brown, Guiding cell migration in 3D: a collagen matrix with graded directional stiffness, Cell Motil Cytoskeleton 66(3) (2009) 121-8.
[50] A. Rizwan, M. Cheng, Z. M. Bhujwalla, B. Krishnamachary, L. Jiang, K. Glunde, Breast cancer cell adhesome and degradome interact to drive metastasis, NPJ Breast Cancer 1 (2015) 15017.
[51] D. L. Holliday, V. Speirs, Choosing the right cell line for breast cancer research, Breast Cancer Res 13(4) (2011) 215.
[52] J. I. Herschkowitz, K. Simin, V. J. Weigman, I. Mikaelian, J. Usary, Z. Hu, K. E. Rasmussen, L. P. Jones, S. Assefnia, S. Chandrasekharan, M. G. Backlund, Y Yin, A. I. Khramtsov, R. Bastein, J. Quackenbush, R. I. Glazer, P. H. Brown, J. E. Green, L. Kopelovich, P. A. Furth, J. P. Palazzo, O. I. Olopade, P. S. Bernard, G. A. Churchill, T. Van Dyke, C. M. Perou, Identification of conserved gene expression features between murine mammary carcinoma models and human breast tumors, Genome Biol 8(5) (2007) R76.
[53] J. Xue, J. Xie, W. Liu, Y. Xia, Electrospun Nanofibers: New Concepts, Materials, and Applications, Acc Chem Res 50(8) (2017) 1976-1987.
[54] A. Tirella, T. Liberto, A. Ahluwalia, Riboflavin and collagen: New crosslinking methods to tailor the stiffness of hydrogels, Mater Lett 74 (2012) 58-61.
[55] S. Itoh, K. Takakuda, S. Kawabata, Y. Aso, K. Kasai, H. Itoh, K. Shinomiya, Evaluation of cross-linking procedures of collagen tubes used in peripheral nerve repair, Biomaterials 23(23) (2002) 4475-4481.
[56] J. Wu, Y Hong, Enhancing cell infiltration of electrospun fibrous scaffolds in tissue regeneration, Bioact Mater 1(1) (2016) 56-64.
[57] T. G. Kim, H. J. Chung, T. G. Park, Macroporous and nanofibrous hyaluronic acid/collagen hybrid scaffold fabricated by concurrent electrospinning and deposition/leaching of salt particles, Acta Biomater 4(6) (2008) 1611-9.
[58] M. C. Phipps, W. C. Clem, J. M. Grunda, G. A. Clines, S. L. Bellis, Increasing the pore sizes of bone-mimetic electrospun scaffolds comprised of polycaprolactone, collagen I and hydroxyapatite to enhance cell infiltration, Biomaterials 33(2) (2012) 524-34.
[59] S. Yaman, M. Anil-Inevi, E. Ozcivici, H. C. Tekin, Magnetic Force-Based Microfluidic Techniques for Cellular and Tissue Bioengineering, Front Bioeng Biotechnol 6 (2018) 192.
[60] C. Chaubaroux, F. Perrin-Schmitt, B. Senger, L. Vidal, J. C. Voegel, P. Schaaf, Y. Haikel, F. Boulmedais, P. Lavalle, J. Hemmerle, Cell Alignment Driven by Mechanically Induced Collagen Fiber Alignment in Collagen/Alginate Coatings, Tissue Eng Part C Methods 21(9) (2015) 881-8.
[61] M. Julias, L. T. Edgar, H. M. Buettner, D. I. Shreiber, An in vitro assay of collagen fiber alignment by acupuncture needle rotation, Biomed Eng Online 7 (2008) 19.
[62] J. A. M. Nuhn, A. M. Perez, I. C. Schneider, Contact guidance diversity in rotationally aligned collagen matrices, Acta Biomater 66 (2018) 248-257.
[63] A. Ray, O. Lee, Z. Win, R. M. Edwards, P. W. Alford, D. H. Kim, P. P. Provenzano, Anisotropic forces from spatially constrained focal adhesions mediate contact guidance directed cell migration, Nat Commun 8 (2017) 14923.
[64] K. H. Nam, P. Kim, D. K. Wood, S. Kwon, P. P. Provenzano, D. H. Kim, Multiscale Cues Drive Collective Cell Migration, Sci Rep 6 (2016) 29749.
[65] O. Ilina, P. G. Gritsenko, S. Syga, J. Lippoldt, C. A. M. La Porta, O. Chepizhko, S. Grosser, M. Vullings, G. J. Bakker, J. Starruss, P. Bult, S. Zapperi, J. A. Kas, A. Deutsch, P. Friedl, Cell-cell adhesion and 3D matrix confinement determine jamming transitions in breast cancer invasion, Nat Cell Biol 22(9) (2020) 1103-1115.
[66] H. T. Nia, L. L. Munn, R. K. Jain, Physical traits of cancer, Science 370(6516) (2020).
[67] V. Padmanaban, I. Krol, Y. Suhail, B. M. Szczerba, N. Aceto, J. S. Bader, A. J. Ewald, E-cadherin is required for metastasis in multiple models of breast cancer, Nature 573(7774) (2019) 439-444.
[68] N. Aceto, A. Bardia, D. T. Miyamoto, M. C. Donaldson, B. S. Wittner, J. A. Spencer, M. Yu, A. Pely, A. Engstrom, H. Zhu, B. W. Brannigan, R. Kapur, S. L. Stott, T. Shioda, S. Ramaswamy, D. T. Ting, C. P. Lin, M. Toner, D. A. Haber, S. Maheswaran, Circulating tumor cell clusters are oligoclonal precursors of breast cancer metastasis, Cell 158(5) (2014) 1110-1122.
[69] K. J. Cheung, V. Padmanaban, V. Silvestri, K. Schipper, J. D. Cohen, A. N. Fairchild, M. A. Gorin, J. E. Verdone, K. J. Pienta, J. S. Bader, A. J. Ewald, Polyclonal breast cancer metastases arise from collective dissemination of keratin 14-expressing tumor cell clusters, Proc Natl Acad Sci USA 113(7) (2016) E854-63.
[70] A. Lugli, R. Kirsch, Y. Ajioka, F. Bosman, G. Cathomas, H. Dawson, H. El Zimaity, J. F. Flejou, T. P. Hansen, A. Hartmann, S. Kakar, C. Langner, I. Nagtegaal, G. Puppa, R. Riddell, A. Ristimaki, K. Sheahan, T. Smyrk, K. Sugihara, B. Terris, H. Ueno, M. Vieth, I. Zlobec, P. Quirke, Recommendations for reporting tumor budding in colorectal cancer based on the International Tumor Budding Consensus Conference (ITBCC) 2016, Mod Pathol 30(9) (2017) 1299-1311.
[71] F. J. Gujam, D. C. McMillan, Z. M. Mohammed, J. Edwards, J. J. Going, The relationship between tumour budding, the tumour microenvironment and survival in patients with invasive ductal breast cancer, Br J Cancer 113(7) (2015) 1066-74.
[72] K. O'Connor, H. H. Li-Chang, S. E. Kalloger, R. D. Peixoto, D. L. Webber, D. A. Owen, D. K. Driman, R. Kirsch, S. Serra, C. H. Scudamore, D. J. Renouf, D. F. Schaeffer, Tumor budding is an independent adverse prognostic factor in pancreatic ductal adenocarcinoma, Am J Surg Pathol 39(4) (2015) 472-8.
[73] V. H. Koelzer, R. Langer, I. Zlobec, A. Lugli, Tumor budding in upper gastrointestinal carcinomas, Front Oncol 4 (2014) 216.
[74] M. Rokutan-Kurata, A. Yoshizawa, N. Nakajima, Y. Teramoto, S. Sumiyoshi, K. Kondo, M. Hamaji, M. Sonobe, T. Menju, H. Date, H. Haga, Discohesive growth pattern (Disco-p) as an unfavorable prognostic factor in lung adenocarcinoma: an analysis of 1062 Japanese patients with resected lung adenocarcinoma, Mod Pathol (2020).
[75] A. Almangush, T. Salo, J. Hagstrom, I. Leivo, Tumour budding in head and neck squamous cell carcinoma—a systematic review, Histopathology 65(5) (2014) 587-94.
[76] H. Ueno, M. Ishiguro, E. Nakatani, T. Ishikawa, H. Uetake, Matsuda, Y. Nakamoto, M. Kotake, K. Kurachi, T. Egawa, K. Yasumasa, K. Murata, O. Ikawa, S. Shinji, K. Murotani, S. Matsui, S. Teramukai, N. Tomita, K. Sugihara, S. S. Group, Prospective Multicenter Study on the Prognostic and Predictive Impact of Tumor Budding in Stage II Colon Cancer: Results From the SACURA Trial, J Clin Oncol 37(22) (2019) 1886-1894.
[77] A. D. Grigore, M. K. Jolly, D. Jia, M. C. Farach-Carson, H. Levine, Tumor Budding: The Name is EMT. Partial EMT, J Clin Med 5(5) (2016).
[78] A. Haeger, M. Krause, K. Wolf, P. Friedl, Cell jamming: collective invasion of mesenchymal tumor cells imposed by tissue confinement, Biochim Biophys Acta 1840(8) (2014) 2386-95.
[79] A. S. Piotrowski-Daspit, B. A. Nerger, A. E. Wolf, S. Sundaresan, C. M. Nelson, Dynamics of Tissue-Induced Alignment of Fibrous Extracellular Matrix, Biophys J 113(3) (2017) 702-713.
[80] X. Liu, R. Taftaf, M. Kawaguchi, Y. F. Chang, W. Chen, D. Entenberg, Y. Zhang, L. Gerratana, S. Huang, D. B. Patel, E. Tsui, V. Adomo-Cruz, S. M. Chirieleison, Y. Cao, A. S. Harney, S. Patel, A. Patsialou, Y. Shen, S. Avril, H. L. Gilmore, J. D. Lathia, D. W. Abbott, M. Cristofanilli, J. S. Condeelis, H. Liu, Homophilic CD44 Interactions Mediate Tumor Cell Aggregation and Polyclonal Metastasis in Patient-Derived Breast Cancer Models, Cancer Discov 9(1) (2019) 96-113.
[81] C. Zeltz, D. Gullberg, The integrin-collagen connection—a glue for tissue repair?, J Cell Sci 129(4) (2016) 653-64.
[82] S. Chen, S. K. Boda, S. K. Batra, X. Li, J. Xie, Emerging Roles of Electrospun Nanofibers in Cancer Research, Adv Healthc Mater 7(6) (2018) e1701024.
[83] M. T. Nelson, A. Short, S. L. Cole, A. C. Gross, J. Winter, T. D. Eubank, J. J. Lannutti, Preferential, enhanced breast cancer cell migration on biomimetic electrospun nanofiber ‘cell highways’, BMC Cancer 14 (2014) 825.

TABLE 1
Previous tumor models with the predisposed ECM structure

| | | | | |
|---|---|---|---|---|
| Techniques to pre- | | | | |
| align ECM fibers | Cell models | Findings | Limitations | References |

Multicellular spheroid

| | | | | |
|---|---|---|---|---|
| Cellular contraction | MDA-MB-231 | Cell spheroids | Microcavities used to | [34] |
| of adjacent cell | spheroids seeded in | apply tensile force | embed spheroids may | |
| spheroids | microcavities | to align ECM fibers | interrupt the predisposed | |
| | | which in turn guide | ECM structure | |
| | | collective | Fiber alignment in | |
| | | migration | restricted areas | |

Single cell

| | | | | |
|---|---|---|---|---|
| Cellular contraction | MDA-MB-231 | Aligned fibers | Cancer cell-laden hydrogel | [18, 19] |
| of fibroblasts | individual cell- | enhance the | was attached adjacent to | |
| followed by | laden hydrogel | directional | but not seeded within pre- | |
| decellularization | attached to pre- | migration of cancer | aligned fibers | |
| | aligned collagen | stem cells | Decellularization may | |
| | gel | | damage the predisposed | |
| | | | collagen fiber structure | |
| Confined geometry | MDA-MB-231 | Cancer cells | Challenging to be applied | [9] |
| and vacuum flow | individual cell pre- | migration in | as a tumor spheroid model | |
| guidance in | seeded in collagen | aligned fibers is | because pre-seeded tumor | |
| microfluidics | gel before | Rho-independent | spheroids may interfere | |
| | alignment | | with the alignment | |
| Injection force into | MDA-MB-231 and | Aligned fibers | methods | [20] |
| hydrogel interface | MCF7 individual | enhance cancer cell | | |
| | cells pre-seeded in | intravasation | | |
| | collagen gel before | | | |
| | alignment | | | |
| Microfluidics- | MDA-MB-231 and | Microscale | | [21] |
| driven shear flow | HCT-116 | collagen bundles | | |
| | individual cells | enhance cancer cell | | |
| | pre-seeded in | migration | | |
| | collagen gel before | | | |
| | alignment | | | |
| Needle rotation | MDA-MB-231 and | Cancer cells | Nonhomogeneous | [61, 62] |
| strain aligns fibers | MTLn3 individual | migration | directionality of fiber | |
| after collagen | cell pre-seeded in | directionality in | alignment | |
| gelling | collagen gel | aligned fibers is | | |
| | | ROCK dependent | | |
| Electrospinning | Cancer cells or | Aligned fibers | Difficult to embed tumor | [35, 82, 83] |
| | tumor spheroids | enhanced cancer | spheroids in high-density | |
| | seeded onto | cell migration | electrospun fibers | |
| | electrospun | | | |
| | scaffolds | | | |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

1. A method for producing a cellular or tissue model, comprising:
applying a phase-specific and force-guided polymerization of a biomaterial, wherein the polymerization comprises a nucleation phase and an elongation phase; thereby,
producing a model.

2. The method of claim 1 further comprising introducing the biomaterial into a polymerization system.

3. The method of claim 1 wherein the system comprises coaxial rotating cylinder system.

4. The method of claim 1 wherein in the nucleation phase, a horizontal laminar Couette flow is generated.

5. The method of claim 1 wherein in the nucleation phase, a horizontal laminar Couette flow is generated by rotating an inner cylinder of the system to promote the adsorption of the biomaterial monomers onto the inner cylinders surface to form an initial coating.

6. The method of claim 1 wherein in the elongation phase, a gravitational force is applied to the biomaterial.

7-8. (canceled)

9. The method of claim 1 wherein the biomaterial comprises collagen I, collagen IV, Matrigel, poly L-lysine, Geltrex, gelatin, nitrogen, fibronectin, fibrinogen, gelatin methacrylate, fibrin, silk, pegylated gels, collagen methacrylate, decellularized extracellular matrices, basement membrane proteins, or a combination thereof.

10-15. (canceled)

16. A tumor model comprising:
a tumor cell or organoid embedded in or otherwise associated with a matrix comprising radially aligned fibers and circumferentially oriented or aligned fibers.

17. The tumor model of claim 16 wherein the tumor cell or organoid are surrounded by radially aligned fibers on one side and circumferentially oriented or aligned fibers the opposing side.

18-21. (canceled)

22. A cell or tissue model comprising:
cells or tissue organoids derived from kidney, liver, brain, heart, lung or stomach embedded in or otherwise associated with a matrix comprising radially aligned fibers and circumferentially oriented or aligned fibers.

23. The model of claim 22 wherein the cells or organoids are surrounded by radially aligned fibers on one side and circumferentially oriented or aligned fibers the opposing side.

24. The model of claim 22 wherein the matrix comprises collagen I, collagen IV, Matrigel, poly L-lysine, Geltrex, gelatin, nitrogen, fibronectin, fibrinogen, gelatin methacrylate, fibrin, silk, pegylated gels, collagen methacrylate, decellularized extracellular matrices, basement membrane proteins, or a combination thereof.

25-27. (canceled)

28. A coaxial rotating cylinder system, comprising:
an outer cylinder;
an inner cylinder;
wherein the outer and inner cylinders comprise different radii and are concentrically aligned.

29-39. (canceled)

40. A method for producing a model, comprising:
employing a coaxial rotating cylinder system of claim 28 and introducing a biomaterial into the empty annulus between the outer and inner cylinders;
applying a phase-specific and force-guided polymerization of the biomaterial, wherein the phase specific polymerization comprises a nucleation phase and an elongation phase; thereby,
producing a model.

41-50. (canceled)

51. A method of distinguishing tumors with different invasive and metastatic potentials, comprising, seeding the system of claim 1 with different tumor cells and analyzing invasion patterns, border complexity and disseminated cell cluster number of each of the different tumor cells.

52. A method of diagnosing cancer, comprising seeding the system of claim 1 with cells from a subject's biological sample; and administering a chemotherapeutic agent to a subject diagnosed as having cancer.

53. A method of screening for candidate therapeutic agents, comprising seeding the system of claim 1 with one or more tumor cells; adding a candidate therapeutic agent to the tumor cells.

54-56. (canceled)

57. A method of screening for candidate therapeutic agents, comprising seeding the system of claim 1 with cells or tissue organoids derived from kidney, heart, lung or stomach; adding a candidate therapeutic agent to cells or tissue organoids derived from kidney, heart, lung or stomach.

58-59. (canceled)

60. A method for treating a subject for cancer, comprising:
seeding the system of claim 1 with one or more tumor cells;
adding a candidate therapeutic agent to the tumor cells;
determining effects of the candidate therapeutic agent on the tumor cells; and
selectively administering the candidate therapeutic agent to the subject.

61-62. (canceled)

64. A method of screening for candidate therapeutic agents, comprising seeding the system of claim 1 with one or more tumor cells; adding a candidate therapeutic agent to determine effects on cell death, invasion patterns, border complexity and disseminated cell cluster number of each of the different tumor cells.
